SEQUENCE LISTING (2 sequences)

SEQ ID NO: 1 - RNA, 1630 nt, Artificial Sequence
  Synthetic polynucleotide
auggaagaugccaaaaacauuaagaagggcccagcgccauucuacccacucgaagacgggaccgccggcgagcagcugcacaaagccaugaagcgcuacgcccuggugcccggcaccaucgccuuuagacgcacauaucgagguggacauuaccuacgccgaguacuucgagaugagcguucggcuggcagaagcuaugaagcgcuaugggcugaauacaaaccaucggaucguggugugcagcgagaauagcucaguucuucaugcccguguugggugcccuguucaucgguguggcuguggccccagcuaacgacaucuacaacgagcgcgagcugcugaacagcaugggcaucagccagcccaccgucguauucguagaagaaagggcugcaaaagauccucaacgugcaaaagaagcuaccgaucauacaaaagaucaucaucauggauagcaagaccgacuaccagggcuuccaaagcauguacaccuucgugacuucccuuuccacccggcuucaacgaguacgacuucgugcccgagagcuucgaccgggacaaaaccaucgcccugaucaugaacaguaguggcaguaccggauugcccaagggcguagcccuaccgcaccgcccgcuguguccgauucagucaugcccgcgaccccaucuucggcaaccagaucauccccgacaccgcuauccucagcguggugccauuucaccacggcuucggcauguucaccacgcugggcuacuuaucugggcuuucgggucgugcucauguaccgcuucgaggaggagcuauucuugcgcagcuugcaagacuauaagauucaaucugcccugcuggugcccacacuauuuagcuucuucgcuaagagcaucucaugacaaguacgaccuaagcaacuugcacgagaucgccagcggcggggcgccgcucagcaaggagguaggugaggccguggccaaacgcuuccaccuaccaggcauccgccagggcuacggcugacagaacaaccagcgccauucugaucacccccgaaggggacgacaagccuggcgcaguaggcaagguggugcccuucuucgaggcuaaggugguggacuuggacaccgguaagacacuggguguaaccagcgggcgagcugugcguccguggccccaugaucaugagcggcuacguuaacaaccccgaggcuacaaacgcucucaucgacaaggacggcuggcugcacagcggcgacaucgccuacugggcgaggacgagcacuucuucaucguggaccggcugaagagccugaucaaauacaagggcuaccagguagccccagccgaacuggagagcauccugcugcaacaccccaacaucuucgacgccggggucgggccugcccgacgacgaugccggcgagcugcccgccgcagucgucgugcuggaacacgguaaaaccaugaccgagaaggagaucguggacuauguggccagccagguuacaaccgccaagaagcuggggugguguuguguucguggacgaggugccuaaaggacugaccggcaaguuggacgcccgcaagauccgcgagauucucauuaaggccaagaagggcggcaagaucgccguguaa

SEQ ID NO: 2 - DNA, 888 bp, Artificial Sequence
  Synthetic polynucleotide
taacggccacctaccttatagatcggcagccttaacaattcccaaccttctcgtgctcgtgattggccccccatgagcattcaataagagggtacacgggtcttggtcaaggtcagagagaaacagaaatgcgatcccgctgagtcaccgatatccgttggccagccagtgcccgttgggagtccgcacagcaaagagcgcttcgtcacacgaaacgggcaaattgtatcagatgaacgccttccgcaaagacgacgcgccacaaggaagctgagctctaaggttaccaataacggctgtaggcccctagctttctcctgcgcccaaataaccgctaacgcggctatgcctcgcacggttttaacttggtacgggcatctcagatttggcgccccggattagcatctcgggcgcttcagcccggcatctataaaggagagccacaccgagatcccgtggagtgccagatcgtttcgcataagggggggccgactgggggaagcgccaagacggggcggcggccgcagccaatgtacccggctcaaatgccttatccgtgccctgacacgaccaacaagtgccgagaagctatgccgggtatagcgacacccgcgaacgattacggtgcgactagcattattacctgctcatggggcaactcaacttgaacaactcatccctccgccccgcagcccgctagatactggagatattcagcacaggaagcgctcctgagctttcactccctgctgggggggccaatatccataaaggacctgagtggagatatccgggcagccggactgggggacgtgacgacaactatacgcagatttagccgccgagggcccgcgaaagccgggtcaccgcccccttctatctccctag

(19) United States
(12) Patent Application Publication
(10) Pub. No.: US 2019/0194390 A1
(43) Pub. Date: Jun. 27, 2019

(54) AMINO-POLYESTERS FOR DRUG DELIVERY

(71) Applicants: Massachusetts Institute of Technology, Cambridge, MA (US); POLITECNICO DI MILANO, Milan (IT)

(72) Inventors: Umberto Capasso Palmiero, Zürich (CH); Piotr S. Kowalski, Allston, MA (US); Davide Moscatelli, Arese (IT); Daniel Griffith Anderson, Framingham, MA (US)

(21) Appl. No.: 16/179,714

(22) Filed: Nov. 2, 2018

Related U.S. Application Data
(60) Provisional application No. 62/678,795, filed on May 31, 2018.
(60) Provisional application No. 62/581,285, filed on Nov. 3, 2017.

(51) Int. Cl. C08G 63/91; A61K 31/7105; A61K 47/34

(52) U.S. Cl. CPC C08G 63/912; A61K 47/34; A61K 31/7105

(57) ABSTRACT

Disclosed are methods, compositions, reagents, systems, and kits to prepare and utilize amino-polyesters (APEs). The APEs are shown to be effective biodegradable carriers for drug delivery applications.

### RELATED APPLICATIONS

This application claims priority under 35 U.S.C. § 119(e) to U.S. Provisional Application, U.S. Ser. No. 62/581,285, filed on Nov. 3, 2017, and to U.S. Provisional Application, U.S. Ser. No. 62/678,795, filed on May 31, 2018, each of which is incorporated herein by reference in their entirety.

### GOVERNMENT SUPPORT

This invention was made with Government support under Grant No. W32P4Q-13-1-0011 awarded by the Defense Advanced Research Projects Agency. The Government has certain rights in the invention.

### BACKGROUND OF THE INVENTION

Polymer biomaterials have been widely used in the development of delivery systems for a variety of therapeutic payloads, including small molecules, proteins, and nucleic acids (e.g., DNA, antisense oligonucleotides (ASO), short interfering RNA (siRNA), micro RNA (miRNA), and messenger RNA (mRNA)).¹ Delivery of RNA-based therapeutics, in particular, allows for both transient control of protein and gene expression and permanent editing of the genomic DNA, providing a therapeutic platform suitable for addressing a wide range of diseases.² Cationic polymers are among the most studied materials for nucleic acid delivery given their ability to electrostatically condense nucleic acids into nanoparticles.³ Several cationic polymers, such as alkyl-functionalized low molecular weight polyethyleneimine (7c1),⁵ poly(amido-amine) (PAMAM) dendrimer-lipid derivatives, and poly-beta amino-esters (PBAEs)⁶, have been employed for in vivo delivery of therapeutic cargos; however, limited efficacy and concerns regarding the toxicity of the above and many other cationic polymers still remain.^{7,8} High net-positive charge, and the inability to undergo degradation under physiological conditions (e.g., 7c1, PAMAM dendrimers), or potentially toxic degradation products (e.g., derivatives of bisphenol A for PBAE), which can accumulate in the body at hazardous levels, are the main problems that limit clinical applications of such biomaterials. In addition, obtaining defined polymers with a narrow molecular weight distribution (with D values close to 1) using step growth polymerization (e.g. Michael addition), polycondensation, or nucleophilic substitution of epoxides is difficult, leading to high batch-to-batch variability, and requiring complicated purification and characterization procedures.

Ring opening polymerization (ROP) has emerged as a versatile method for the synthesis of polymers with controlled molecular weight and narrow polydispersity.⁹ Notably, polyesters prepared by ROP of lactones and lactides, including polycaprolactone (PCL), polylactide (PLA), and polyglycolic acid (PLGA), have been widely used clinically due to their superior biocompatibility and degradability.¹⁰ Adopting ROP for the synthesis of amine-containing polyesters for nucleic acid delivery, however, involves several synthetic challenges related to incompatibility of primary and secondary amines with the ROP, as well as the lack of naturally occurring amine bearing lactone monomers. The few published methods involve multi-step syntheses requiring protection/deprotection after polymerization, or post-polymerization modification of functionalized polyesters. Both approaches suffer from problems with conversion, scalability, and polymer-chain degradation.¹¹ Methods utilizing ROP of amine-containing lactone monomers present an elegant synthetic strategy for preparation of amino polyesters but require multi-step synthesis of functional monomers.¹² In addition, it is difficult to predict a priori the biological compatibility of these polymers, and their corresponding degradation products, such as lipocationic hydroxy acids.^{12,13} In contrast, polyester derivatives of readily available lactones that are often found in natural products or used in food and biomedical applications, are known to possess low toxicity and are generally recognized as safe substances (GRAS) by an American Food and Drug Administration (FDA). Such lactones have been used to prepare poly(amine-co-esters) capable of DNA transfection via combined enzymatic ring opening and polycondensation copolymerization of lactones in the presence of tertiary amines and dialkyl-diester catalyzed by Candida antarctica lipase B (CALB).¹⁴ Despite these advances, there remains a need for new amino-polyesters and methods for making them.

### SUMMARY OF THE INVENTION

The present disclosure describes the synthesis of a new type of ionizable amino-polyesters (APEs) via controlled ROP of lactones with tertiary amino-alcohols. Such APEs are synthesized using a one step synthesis method. The method provides control over the number of repeating monomer units (q) by varying the stoichiometry between the alkoxy bearing initiator and the lactone monomer, yielding degradable polymers with a narrow polydispersity ((Ð)<1.4) in high yields. A study of the structure-activity relationships, and the ability of APEs to promote in vivo delivery of mRNA is disclosed. As such, the present disclosure addresses challenges of current drug delivery systems.

Amino-polyesters, also referred to as amino-polylactones, are provided herein. Methods, compositions, reagents, systems, and kits that allow for the preparation and utilization of amino-polyester compounds are disclosed herein. Methods and reagents to prepare polylactone diacrylates are also disclosed herein.

In one aspect, provided herein is a compound of Formula (I):

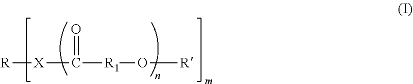

or a pharmaceutically acceptable salt thereof, wherein:

X is O, S, or NR₄;

R is optionally substituted heteroaliphatic, optionally substituted heterocyclyl, or a combination thereof, wherein R comprises one or more amine moieties;

each R₁ independently is optionally substituted aliphatic; optionally substituted carbocyclyl; optionally substituted heteroaliphatic; or optionally substituted heterocyclyl;

R′ is hydrogen, or a group of the formula:

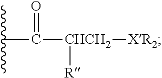

each R″ independently is hydrogen, optionally substituted aliphatic, or optionally substituted heteroaliphatic;

each R₂ independently is hydrogen, optionally substituted aliphatic, optionally substituted heteroaliphatic, optionally substituted carbocyclyl, optionally substituted heterocyclyl, optionally substituted aryl, or optionally substituted heteroaryl;

each X′ independently is O, S, or NR₃;

each R₃ is hydrogen, optionally substituted, aliphatic; optionally substituted carbocyclyl; optionally substituted heteroaliphatic; optionally substituted hetercyclyl; optionally substituted aryl; or optionally substituted heteroaryl; or R₂ and R₃ are combined to form an optionally substituted heterocyclyl;

each R₄ independently is optionally substituted, aliphatic; optionally substituted carbocyclyl; optionally substituted heteroaliphatic; optionally substituted hetercyclyl; optionally substituted aryl; or optionally substituted heteroaryl;

each n independently is an integer between 1 and 20, inclusive; and

m is an integer between 1 and 10, inclusive.

In certain embodiments, the compound of Formula (I) is of Formula (II):

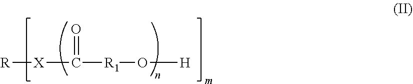

or a pharmaceutically acceptable salt thereof.

In certain embodiments, the compound of Formula (III):

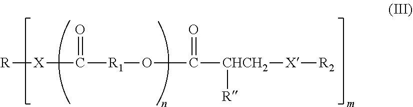

or a pharmaceutically acceptable salt thereof.

In other aspects, the present disclosure provides methods of preparing compounds described herein. In one aspect, provided herein are methods of making a compound of Formula (II), or a salt thereof, comprising acylating a compound of Formula (Ib),

R(X)ₘ (Ib)

or a salt thereof, wherein R, X and m are as defined herein, with a compound of Formula (Ic),

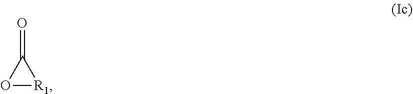

wherein R₁ is as defined herein, to obtain a compound of Formula (II), or a salt thereof.

In another aspect, provided herein is a method of making a compound of Formula (III), or a salt thereof, comprising acylating a compound of Formula (II) to obtain a compound of Formula (IIa),

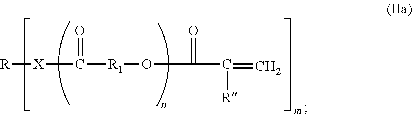

and alkylating a compound of Formula (IIb),

HX′R₂ (IIb)

with the compound of Formula (IIa) to obtain the compound of Formula (III), or a salt thereof.

In some embodiments, the present disclosure provides compositions comprising a polymer described herein and optionally an excipient. In certain embodiments, the composition is a pharmaceutical composition. In certain embodiments, the composition is a nutraceutical composition. In certain embodiments, the composition is a composition with non-medical application.

In certain embodiments, the composition further comprises cholesterol. In certain embodiments, the composition further comprises a PEG derivative (e.g., a PEG moiety conjugate to a hydrophobic moiety, such as a PEGylated lipid, PEG-alkyl, PEG-polycaprolactone, and the like). In certain embodiments, the composition further comprises a phospholipid. In certain embodiments, the composition further comprises an agent.

In certain embodiments, the composition further comprises a zwitterionic molecule, such as a poly(carboxybetaine), poly(sulfobetaine), or poly(2-methacryloyloxyethyl phosphorylcholine).

The present disclosure also provides methods of using compositions described herein. In certain embodiments, the compositions are used to treat a disease, disorder, or condition from which a subject suffers comprising administering to a subject in need thereof an effective amount of a composition described herein. In certain embodiments, the compositions are used to deliver a polymer to a cell comprising contacting the cell with a composition described herein.

In further embodiments, the present disclosure provides kits comprising one or more components selected from compounds described herein.

The details of certain embodiments of the invention are set forth in the Detailed Description of Certain Embodiments, as described below. Other features, objects, and advantages of the invention will be apparent from the Definitions, Figures, Examples, and Claims.

### BRIEF DESCRIPTION OF THE DRAWINGS

FIG. 1. ¹H-NMR and peak assignments of B-DD3 before purification.

FIGS. 2A-2C. (FIG. 2A) Schematic synthesis of the amino-polyesters via ring opening polymerization of lactones initiated by tertiary amino-alcohols. (FIG. 2B) Schematic synthesis of functionalized APE using acryloyl chloride and subsequent end-functionalization via Michael addition with different amines. (FIG. 2C) Exemplary lactones, amino-alcohols, and amine libraries for the synthesis of APEs.

FIGS. 3A-3D. (FIG. 3A) Polyester Mn as a function of the lactone (CL, DD, TD) conversion for the ROP initiated by the amino-alcohol B and with a targeted q=3. (FIG. 3B) ¹H-NMR characterization of the ROP of the CL initiated by the amino-alcohol B and with a targeted q=3 (FIG. 3C) In vitro screening of APE nanoparticles containing Fluc mRNA in Hela cells. Data are presented as mean relative luminescence±SD; n=4. (FIG. 3D) Representative GPC traces for the APEs (I-DD3, A-TD3, and B-DD3) chosen for further in vitro and in vivo studies.

FIGS. 4A-4B. (FIG. 4A) Flow cytometry analysis of the uptake of APE lipid nanoparticles (LNPs) containing Cy5 mRNA. Data are presented as mean±SD; n=3 (FIG. 4B) Representative images of the APE LNPs uptake in HeLa cells. Nuclei are stained with DAPI (Blue), Cy5 mRNA (Red), DIC, differential interference contrast. Scale bar 50 μm.

FIGS. 5A-5B. (FIG. 5A) Cy5 mRNA tissue distribution was analyzed using IVIS (in vitro imaging system). Data are presented as mean±SD; n=3, (FIG. 5B) Representative tissue distribution, Lu—lungs, H—heart, L—liver, S—spleen, K—kidney, P—pancreas.

FIGS. 6A-6B. (FIG. 6A) Flue mRNA expression was analyzed using IVIS. Data are presented as mean±SD; n=3. (FIG. 6B) Representative Flue mRNA expression within the tissues.

FIGS. 7A-7B. (FIG. 7A) Flue mRNA expression was analyzed using IVIS. Data are presented as mean±SD; n=3-4. (FIG. 7B) Representative Flue mRNA expression within the tissues.

FIGS. 8A-8B. (FIG. 8A)¹H-NMR characterization of B-DD3 end-functionalized with acryloyl chloride (Ac) and subsequently with selected amine (1) (FIG. 8B) GPC analysis of the B-CL3 and B-DD3-Ac1.

FIGS. 9A-9D. (FIG. 9A) Characterization of the size of B-DD3 nanoparticles with different formulation (FIG. 9B) In vitro screening of APE nanoparticles containing Flue mRNA in Hela cells. Data are presented as mean relative luminescence±SD; n=4. (FIGS. 9C-9D) Comparison of in vitro mRNA delivery efficacy between differently functionalized and formulated BDD-3 APEs. Data are presented as mean relative luminescence±SD; n=3.

FIGS. 10A-10C. (FIG. 10A) In vivo delivery efficacy of Flue mRNA by B-DD3 nanoparticles formulated as APE:DOPE:Chol:C14-PEG2000 (LNP) Co or APE:C14-PEG2000 (PEG). Data sets show representative IVIS images. (FIG. 10B) Quantification of the luminescence signal from selected organs. Data are presented as mean±SD; n=3. (FIG. 10C) Comparison of the Flue mRNA delivery to the lungs between B-DD3-Ac1, Ac4 (0.7 mg/kg) and 7c1 (non-degradable polymer, 1 mg/kg).

FIG. 11. Examples of lactone monomers that can be used to synthesize amino-polyesters.

FIG. 12. Examples of the initiators that bears tertiary amines and terminal primary/secondary amines that can be used to synthesize amino-polyesters.

FIGS. 13A-13B. (FIG. 13A) Comparison of the Flue mRNA delivery to muscle between B-DD3-Ac1, Ac4 and 7c1. (FIG. 13B) Mice were injected into thigh muscle with APE LNPs containing 5 μg of Flue mRNA and imaged by IVIS after 6 h.

FIG. 14. Comparison of the FLuc expression relative to viability for cells transfected with different concentrations of APE-LNPs formulated with Firefly Luciferase (Fluc) siRNA.

FIG. 15. Comparison of the luminescence fold-induction for cells transfected with different concentrations of APE-LNPs formulated with plasmid DNA encoding Firefly Luciferase (Fluc).

FIG. 16. Schematic synthesis of functionalized APE (top). Exemplary lactones, amino-alcohols, and amine libraries for the synthesis of APEs (bottom.)

FIG. 17. Comparison of in vitro mRNA delivery efficacy between differently functionalized and formulated B-CL APEs. Functionalized B-CL LNPs show 160-fold increase in Fluc mRNA expression.

FIG. 18. Comparison of in vitro mRNA delivery efficacy between differently functionalized and formulated A-TD APEs. Functionalized A-TD LNPs show 56-fold increase in Fluc mRNA expression.

FIG. 19. Comparison of in vitro mRNA delivery efficacy between differently functionalized and formulated B-DD APEs. Functionalized B-DD LNPs show 73-fold increase in Fluc mRNA expression.

FIGS. 20A-20B. In vivo evaluation of functionalized versus non-functionalized APEs. (FIG. 20A) Quantification of FLuc mRNA B-DD3 and B-DD3_A5 expression in the spleen tissue. Data are presented as mean±SD; n=3; * p<0.05. (FIG. 20B) Representative IVIS images of FLuc mRNA expression within the tissues.

### DEFINITIONS

For convenience, certain terms employed herein, in the specification, examples and appended claims are collected herein.

Unless otherwise required by context, singular terms shall include pluralities, and plural terms shall include the singular.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can include one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

In a formula, [symbol] is a single bond where the stereochemistry of the moieties immediately attached thereto is not specified, - - - is absent or a single bond, and [symbol] or [symbol] is a single or double bond.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The term “aliphatic” includes both saturated and unsaturated, straight chain (i.e., unbranched), branched, acyclic, cyclic, or polycyclic aliphatic hydrocarbons, which are optionally substituted with one or more functional groups. Likewise, the term “heteroaliphatic” refers to heteroalkyl, heteroalkenyl, heteroalkynyl, and heterocyclic groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-100 aliphatic carbon atoms. In certain other embodiments, the alkyl, alkenyl, and alkynyl groups employed in the disclosure contain 1-50 aliphatic carbon atoms. In certain other embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, npropyl, isopropyl, cyclopropyl, —CH₂— cyclopropyl, vinyl, allyl, n-butyl, sec-butyl, isobutyl, tertbutyl, cyclobutyl, —CH₂— cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties, and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” refers to a radical of a straight-chain or branched saturated hydrocarbon group. In some embodiments, an alkyl group has 1 to 1000 carbon atoms (“C₁-C₁₀₀₀ alkyl”), 1 to 900 carbon atoms (“C₁-C₉₀₀ alkyl”), 1 to 800 carbon atoms (“C₁-C₈₀₀ alkyl”), 1 to 700 carbon atoms (“C₁-C₇₀₀ alkyl”), 1 to 600 carbon atoms (“C₁-C₆₀₀ alkyl”), 1 to 500 carbon atoms (“C₁-C₅₀₀ alkyl”), 1 to 400 carbon atoms (“C₁-C₄₀₀ alkyl”), 1 to 300 carbon atoms (“C₁-C₃₀₀ alkyl”), 1 to 200 carbon atoms (“C₁-C₂₀₀ alkyl”), 1 to 100 carbon atom (“C₁-C₁₀₀ alkyl”). In some embodiments, an alkyl group has 1 to 10 carbon atoms (“C₁-C₁₀ alkyl”), 1 to 9 carbon atoms (“C₁-C₉ alkyl”), 1 to 8 carbon atoms (“C₁-C₈ alkyl”), 1 to 7 carbon atoms (“C₁-C₇ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), 1 to 2 carbon atoms (“C₁-C₂ alkyl”), or 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents.

The term “alkenyl” refers to a radical of a straight-chain or branched hydrocarbon group having from 2 to 1000 carbon atoms and one or more carbon-carbon double bonds (e.g., 1, 2, 3, or 4 double bonds). In some embodiments, an alkenyl group has 2 to 1000 carbon atoms (“C₂-C₁₀₀₀ alkenyl”), 2 to 900 carbon atoms (“C₂-C₉₀₀ alkenyl”), 2 to 800 carbon atoms (“C₂-C₈₀₀ alkenyl”), 2 to 700 carbon atoms (“C₂-C₇₀₀ alkenyl”), 2 to 600 carbon atoms (“C₂-C₆₀₀ alkenyl”), 2 to 500 carbon atoms (“C₂-C₅₀₀ alkenyl”), 2 to 400 carbon atoms (“C₂-C₄₀₀ alkenyl”), 2 to 300 carbon atoms (“C₂-C₃₀₀ alkenyl”), 2 to 200 carbon atoms (“C₂-C₂₀₀ alkenyl”), 2 to 100 carbon atom (“C₂-C₁₀₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified (e.g., —CH═CHCH₃,

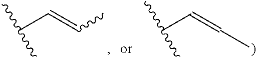

may be in the (E)- or (Z)-configuration.

The term “alkynyl” refers to a radical of a straight-chain or branched hydrocarbon group having from 2 to 1000 carbon atoms and one or more carbon-carbon triple bonds (e.g., 1, 2, 3, or 4 triple bonds). In some embodiments, an alkynyl group has 2 to 1000 carbon atoms (“C₂-C₁₀₀₀ alkynyl”), 2 to 900 carbon atoms (“C₂-C₉₀₀ alkynyl”), 2 to 800 carbon atoms (“C₂-C₈₀₀ alkynyl”), 2 to 700 carbon atoms (“C₂-C₇₀₀ alkynyl”), 2 to 600 carbon atoms (“C₂-C₆₀₀ alkynyl”), 2 to 500 carbon atoms (“C₂-C₅₀₀ alkynyl”), 2 to 400 carbon atoms (“C₂-C₄₀₀ alkynyl”), 2 to 300 carbon atoms (“C₂-C₃₀₀ alkynyl”), 2 to 200 carbon atoms (“C₂-C₂₀₀ alkynyl”), 2 to 100 carbon atom (“C₂-C₁₀₀ alkynyl”). In some embodiments, an alkynyl group has 2 to 9 carbon atoms (“C₂₋₉ alkynyl”), 2 to 8 carbon atoms (“C₂₋₈ alkynyl”), 2 to 7 carbon atoms (“C₂₋₇ alkynyl”), 2 to 6 carbon atoms (“C₂₋₆ alkynyl”), 2 to 5 carbon atoms (“C₂₋₅ alkynyl”), 2 to 4 carbon atoms (“C₂₋₄ alkynyl”), 2 to 3 carbon atoms (“C₂₋₃ alkynyl”), or 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Unless otherwise specified, each instance of an alkynyl group is independently unsubstituted (an “unsubstituted alkynyl”) or substituted (a “substituted alkynyl”) with one or more substituents.

The term “heteroalkyl” refers to an alkyl group which further includes at least one heteroatom (e.g., 1, 2, 3, or 4 heteroatoms) selected from oxygen, nitrogen, phosphorus, or sulfur within (i.e., inserted between adjacent carbon atoms of) and/or placed at one or more terminal position(s) of the parent chain. In certain embodiments, a heteroalkyl group refers to a saturated group having from 1 to 1000 carbon atoms and 1 or more heteroatoms within the parent chain (“C₁-C₁₀₀₀ heteroalkyl”), 1 to 900 carbon atoms and 1 or more heteroatoms within the parent chain (“C₁-C₉₀₀ heteroalkyl”), 1 to 800 carbon atoms and 1 or more heteroatoms within the parent chain (“C₁-C₈₀₀ heteroalkyl”), 1 to 700 carbon atoms and 1 or more heteroatoms within the parent chain (“C₁-C₇₀₀ heteroalkyl”), 1 to 600 carbon atoms and 1 or more heteroatoms within the parent chain (“C₁-C₆₀₀ heteroalkyl”), 1 to 500 carbon atoms and 1 or more heteroatoms within the parent chain (“C₁-C₅₀₀ heteroalkyl”), 1 to 400 carbon atoms and 1 or more heteroatoms within the parent chain (“C₁-C₄₀₀ heteroalkyl”), 1 to 300 carbon atoms and 1 or more heteroatoms within the parent chain (“C₁-C₃₀₀ heteroalkyl”), 1 to 200 carbon atoms and 1 or more heteroatoms within the parent chain (“C₁-C₂₀₀ heteroalkyl”), or 1 to 100 carbon atoms and 1 or more heteroatoms within the parent chain (“C₁-C₁₀₀ heteroalkyl”). In certain embodiments, a heteroalkyl group refers to a saturated group having from 1 to 10 carbon atoms and 1 or more heteroatoms within the parent chain (“C₁-C₁₀ heteroalkyl”), 1 to 9 carbon atoms and 1 or more heteroatoms within the parent chain (“C₁-C₉ heteroalkyl”), 1 to 8 carbon atoms and 1 or more heteroatoms within the parent chain (“C₁-C₈ heteroalkyl”), 1 to 7 carbon atoms and 1 or more heteroatoms within the parent chain (“C₁-C₇ heteroalkyl”), 1 to 6 carbon atoms and 1 or more heteroatoms within the parent chain (“C₁-C₆ heteroalkyl”), 1 to 5 carbon atoms and 1 or more heteroatoms within the parent chain (“C₁-C₅ heteroalkyl”), 1 to 4 carbon atoms and 1 or more heteroatoms within the parent chain (“C₁-C₄ heteroalkyl”), 1 to 3 carbon atoms and 1 or more heteroatoms within the parent chain (“C₁-C₃ heteroalkyl”), 1 to 2 carbon atoms and 1 heteroatom within the parent chain (“C₁-C₂ heteroalkyl”), or 1 carbon atom and 1 heteroatom (“C₁ heteroalkyl”). Unless otherwise specified, each instance of a heteroalkyl group is independently unsubstituted (an “unsubstituted heteroalkyl”) or substituted (a “substituted heteroalkyl”) with one or more substituents.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a saturated group having from 1 to 1000 carbon atoms and 1 or more heteroatoms within the parent chain (“heteroC₁-C₁₀₀₀ alkenyl”), 1 to 900 carbon atoms and 1 or more heteroatoms within the parent chain (“heteroC₁-C₉₀₀ alkenyl”), 1 to 800 carbon atoms and 1 or more heteroatoms within the parent chain (“heteroC₁-C₈₀₀ alkenyl”), 1 to 700 carbon atoms and 1 or more heteroatoms within the parent chain (“heteroC₁-C₇₀₀ alkenyl”), 1 to 600 carbon atoms and 1 or more heteroatoms within the parent chain (“heteroC₁-C₆₀₀ alkenyl”), 1 to 500 carbon atoms and 1 or more heteroatoms within the parent chain (“heteroC₁-C₅₀₀ alkenyl”), 1 to 400 carbon atoms and 1 or more heteroatoms within the parent chain (“heteroC₁-C₄₀₀ alkenyl”), 1 to 300 carbon atoms and 1 or more heteroatoms within the parent chain (“heteroC₁-C₃₀₀ alkenyl”), 1 to 200 carbon atoms and 1 or more heteroatoms within the parent chain (“heteroC₁-C₂₀₀ alkenyl”), or 1 to 100 carbon atoms and 1 or more heteroatoms within the parent chain (“heteroC₁-C₁₀₀ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”).

Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a saturated group having from 1 to 1000 carbon atoms and 1 or more heteroatoms within the parent chain (“heteroC₁-C₁₀₀₀ alkynyl”), 1 to 900 carbon atoms and 1 or more heteroatoms within the parent chain (“heteroC₁-C₉₀₀ alkynyl”), 1 to 800 carbon atoms and 1 or more heteroatoms within the parent chain (“heteroC₁-C₈₀₀ alkynyl”), 1 to 700 carbon atoms and 1 or more heteroatoms within the parent chain (“heteroC₁-C₇₀₀ alkynyl), 1 to 600 carbon atoms and 1 or more heteroatoms within the parent chain (“heteroC₁-C₆₀₀ alkynyl”), 1 to 500 carbon atoms and 1 or more heteroatoms within the parent chain (“heteroC₁-C₅₀₀ alkynyl”), 1 to 400 carbon atoms and for more heteroatoms within the parent chain (“heteroC₁-C₄₀₀ alkynyl”), 1 to 300 carbon atoms and 1 or more heteroatoms within the parent chain (“heteroC₁-C₃₀₀ alkynyl”), 1 to 200 carbon atoms and 1 or more heteroatoms within the parent chain (“heteroC₁-C₂₀₀ alkynyl”), or 1 to 100 carbon atoms and 1 or more heteroatoms within the parent chain (“heteroC₁-C₁₀₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” or “cycloalkyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”), 3 to 7 ring carbon atoms (“C₃₋₇ carbocyclyl”), 3 to 6 ring carbon atoms (“C₃₋₆ carbocyclyl”), 4 to 6 ring carbon atoms (“C₄₋₆ carbocyclyl”), 5 to 6 ring carbon atoms (“C₅₋₆ carbocyclyl”), or 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, phosphorus, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents.

In some embodiments, a heterocyclyl group is a 5-10 membered non-aromatic ring system having ring carbon atoms and 1-4 ring heteroatoms, wherein each heteroatom is independently selected from nitrogen, oxygen, phosphorus, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, phosphorus, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, phosphorus, and sulfur (“5-6 membered heterocyclyl”). In some embodiments, the 5-6 membered heterocyclyl has 1-3 ring heteroatoms selected from nitrogen, oxygen, phosphorus, and sulfur. In some embodiments, the 5-6 membered heterocyclyl has 1-2 ring heteroatoms selected from nitrogen, oxygen, phosphorus, and sulfur. In some embodiments, the 5-6 membered heterocyclyl has 1 ring heteroatom selected from nitrogen, oxygen, phosphorus, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl, and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl, and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydroyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 r electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group be monovalent or may have more than one point of attachment to another moiety (e.g., it may be divalent, trivalent, etc), although the valency may be specified directly in the name of the group. For example, “triazoldiyl” refers to a divalent triazolyl moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

As understood from the above, alkyl, alkenyl, alkynyl, carbocyclyl, aryl, and heteroaryl groups are, in certain embodiments, optionally substituted. Optionally substituted refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Affixing the suffix “ene” to a group indicates the group is a polyvalent (e.g., bivalent, trivalent, tetravalent, or pentavalent) moiety. In certain embodiments, affixing the suffix “ene” to a group indicates the group is a bivalent moiety.

Exemplary carbon atom substituents include, but are not limited to, halogen, —CN, —NO₂, —N₃, —SO₂H, —SO₃H, —OH, —OR^{a}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted C₁₋₆ alkyl, —ORₐₐ, —SR^{aa}, —N(R^{bb})₂, —CN, —SCN, or —NO₂.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)clhyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenylmethyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, l-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TEMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “thiol” or “thio” refers to the group —SH.

The term “amine” or “amino” refers to the group —NH— or —NH₂, wherein each H is optionally, independently replaced with an alkyl, heteroalkyl, aryl, or heteroaryl group.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-hetero aliphatic amino, mono- or di-alkylamino, mono- or di-hetero alkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, hetero aliphatic amino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutic ally acceptable salts of the compounds of this disclosure include those derived from suitable inorganic and organic acids and bases.

Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(Cw alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The term “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethyHydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

The term “solvent” refers to a substance that dissolves one or more solutes, resulting in a solution. A solvent may serve as a medium for any reaction or transformation described herein. The solvent may dissolve one or more reactants or reagents in a reaction mixture. The solvent may facilitate the mixing of one or more reagents or reactants in a reaction mixture. The solvent may also serve to increase or decrease the rate of a reaction relative to the reaction in a different solvent. Solvents can be polar or non-polar, protic or aprotic. Common solvents useful in the methods described herein include, but are not limited to, acetone, acetonitrile, benzene, benzonitrile, 1-butanol, 2-butanone, butyl acetate, tert-butyl methyl ether, carbon disulfide carbon tetrachloride, chlorobenzene, 1-chlorobutane, chloroform, cyclohexane, cyclopentane, 1,2-dichlorobenzene, 1,2-dichloroethane, dichloromethane (DCM), N,N-dimethylacetamide N,N-dimethylformamide (DMF), 1,3-dimethyl-3,4,5,6-tetrahydro-2-pyrimidinone (DMPU), 1,4-dioxane, 1,3-dioxane, diethylether, 2-ethoxyethyl ether, ethyl acetate, ethyl alcohol, ethylene glycol, dimethyl ether, heptane, n-hexane, hexanes, hexamethylphosphoramide (HMPA), 2-methoxyethanol, 2-methoxyethyl acetate, methyl alcohol, 2-methylbutane, 4-methyl-2-pentanone, 2-methyl-1-propanol, 2-methyl-2-propanol, 1-methyl-2-pyrrolidinone, dimethylsulfoxide (DMSO), nitromethane, 1-octanol, pentane, 3-pentanone, 1-propanol, 2-propanol, pyridine, tetrachloroethylene, tetrahyrdofuran (THF), 2-methyltetrahydrofuran, toluene, trichlorobenzene, 1,1,2-trichlorotrifluoroethane, 2,2,4-trimethylpentane, trimethylamine, triethylamine, N,N-diisopropylethylamine, diisopropylamine, water, o-xylene, and p-xylene.

As used herein, the term “agent” means a molecule, group of molecules, complex or substance administered to an organism for diagnostic, therapeutic, preventative medical, or veterinary purposes. In certain embodiments, the agent is a pharmaceutical agent (e.g., a therapeutic agent, a diagnostic agent, or a prophylactic agent). In certain embodiments, the compositions disclosed herein comprise an agent(s), e.g., a first therapeutic agent (e.g., at least one (including, e.g., at least two, at least three). In some embodiments, the compositions (e.g., macromonomers, conjugates, or particles) can further comprise a second therapeutic agent, a targeting moiety, a diagnostic moiety as described herein.

As used herein, the term “therapeutic agent” includes an agent that is capable of providing a local or systemic biological, physiological, or therapeutic effect in the biological system to which it is applied. For example, a therapeutic agent can act to control tumor growth, control infection or inflammation, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism.

An agent (e.g., a therapeutic agent) can include a wide variety of different compounds, including chemical compounds and mixtures of chemical compounds (e.g., small organic or inorganic molecules) such as drug compounds (e.g., compounds approved for human or veterinary use by the U.S. Food and Drug Administration as provided in the Code of Federal Regulations (CFR)); targeting agents; isotopically labeled chemical compounds; agents useful in bioprocessing; carbohydrates; saccharines; monosaccharides; oligosaccharides; polysaccharides; biological macromolecules (e.g., peptides, proteins, and peptide analogs and derivatives); peptidomimetics; antibodies and antigen binding fragments thereof; nucleic acids (e.g., DNA or RNA); nucleotides; nucleosides; oligonucleotides; antisense oligonucleotides; polynucleotides; nucleic acid analogs and derivatives; nucleoproteins; mucoproteins; lipoproteins; synthetic polypeptides or proteins; small molecules linked to proteins; glycoproteins; steroids; lipids; hormones; vitamins; vaccines; immunological agents; an extract made from biological materials such as bacteria, plants, fungi, or animal cells; animal tissues; naturally occurring or synthetic compositions; and any combinations thereof.

In some embodiments, the agent is in the form of a prodrug. The term “prodrug” refer to a compound that becomes active, e.g., by solvolysis, reduction, oxidation, or under physiological conditions, to provide a pharmaceutically active compound, e.g., in vivo. A prodrug can include a derivative of a pharmaceutically active compound, such as, for example, to form an ester by reaction of the acid, or acid anhydride, or mixed anhydrides moieties of the prodrug moiety with the hydroxyl moiety of the pharmaceutical active compound, or to form an amide prepared by the acid, or acid anhydride, or mixed anhydrides moieties of the prodrug moiety with a substituted or unsubstituted amine of the pharmaceutically active compound. Simple aliphatic or aromatic esters, amides, and anhydrides derived from acidic groups may comprise prodrugs. In some embodiments, the conjugate or particle described herein incorporates one therapeutic agent or prodrug thereof. In some embodiments, the conjugate or particle described herein incorporates more than one therapeutic agents or prodrugs.

In some embodiments, the agent (e.g., a therapeutic agent) is a small molecule. As used herein, the term “small molecule” can refer to compounds that are “natural product-like.” However, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary agents (e.g., a therapeutic agents) in the compositions include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

In some embodiments, exemplary therapeutic agents in the compositions include, but are not limited to, one or more of the agents listed in Paragraph 0148 of U.S. Pat. No. 9,381,253, incorporated by reference herein.

Agents, e.g., therapeutic agents, include the herein disclosed categories and specific examples. It is not intended that the category be limited by the specific examples. Those of ordinary skill in the art will recognize also numerous other compounds that fall within the categories and that are useful according to the present disclosure.

Examples of therapeutic agents include, but are not limited to, antimicrobial agents, analgesics, antinflammatory agents, counterirritants, coagulation modifying agents, diuretics, sympathomimetics, anorexics, antacids and other gastrointestinal agents; antiparasitics, antidepressants, anti-hypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anti-cancer properties, or a combination thereof. Other suitable therapeutic agents include contraceptives and vitamins as well as micro- and macronutrients. Still other examples include antiinfectives such as antibiotics and antiviral agents; analgesics and analgesic combinations; anorexics; antiheimintics; antiarthritics; antiasthmatic agents; anticonvulsants; antidepressants; antidiuretic agents; antidiarrleals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; anti-hypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psycho stimulants; sedatives; and tranquilizers; and naturally derived or genetically engineered proteins, polysaccharides, glycoproteins, or lipoproteins.

In certain instances, the diagnostic agent is an imaging agent or contrast agent. The terms “imaging agent” and “contrast agent” refer to a substance used to enhance the contrast of structures or fluids within the body in medical imaging. It is commonly used to enhance the visibility of blood vessels and the gastrointestinal tract in medical imaging.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Lriedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma pigmentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (ME), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infections causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felly's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions.

Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Amold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

An “effective amount” of a composition described herein refers to an amount sufficient to elicit the desired biological response. An effective amount of a composition described herein may vary depending on such factors as the desired biological endpoint, the pharmacokinetics of the composition, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a composition or pharmaceutical composition described herein in a single dose. In certain embodiments, an effective amount is the combined amounts of a composition or pharmaceutical composition described herein in multiple doses.

A “therapeutically effective amount” of a composition described herein is an amount sufficient to provide a therapeutic benefit in the treatment of a condition or to delay or minimize one or more symptoms associated with the condition. A therapeutically effective amount of a composition means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a composition described herein is an amount sufficient to prevent a condition, or one or more symptoms associated with the condition or prevent its recurrence. A prophylactically effective amount of a composition means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The terms “nucleic acid” or “nucleic acid sequence”, “nucleic acid molecule”, “nucleic acid fragment” or “polynucleotide” are used interchangeably. A polynucleotide molecule is a biopolymer composed of nucleotide monomers covalently bonded in a chain. DNA (deoxyribonucleic acid) and RNA (ribonucleic acid) are examples of polynucleotides with distinct biological function. DNA consists of two chains of polynucleotides, with each chain in the form of a helical spiral. RNA is more often found in nature as a single-strand folded onto itself. Exemplary types of RNA include double-stranded RNA (dsRNA), small interfering RNA (siRNA), short hairpin (shRNA), microRNA (miRNA), messenger RNA (mRNA), antisense RNA, transfer RNA (tRNA), small nuclear RNA (snRNA), and ribosomal RNA (rRNA).

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “RNA interference” or “RNAi” refers to a biological process in which RNA molecules inhibit gene expression or translation, by neutralizing targets mRNA molecules. Since the discovery of RNAi and its regulatory potentials, it has become evident that RNAi has immense potential in suppression of desired genes. RNAi is now known as precise, efficient, stable, and better than antisense technology for gene suppression. Two types of small ribonucleic acids molecules are central to RNA interference: miRNA and siRNA. These small RNAs can bind to mRNA molecules and either increase or decrease their activity (e.g., preventing an mRNA from being translated into a protein). The RNAi pathway is found in many eukaryotes, including animals, and is initiated by the enzyme Dicer, which cleaves long dsRNA molecules into short double-stranded fragments of ˜20 nucleotide siRNAs. Each siRNA is unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in a mRNA molecule and induces cleavage by Argonaute 2 (Ago2), the catalytic component of the RISC complex. In some organisms, this process spreads systematically, despite the initially limited molar concentrations of siRNA.

The term “biodegradable” or “biodegradation” refers to the disintegration of materials by biological means. Organic material can be degraded aerobically or anaerobically. Decomposition of biodegradable substances may include both biological and abiotic steps.

The term “biocompatible” or “biocompatibility” refers to the ability of a material to perform with an appropriate host response in a specific situation. In particular, the terms refer to the ability of a biomaterial to perform its desired function with respect to a medical therapy without eliciting any undesirable local or systematic effects in the recipient or beneficiary of that therapy, but generating the most appropriate beneficial cellular or tissue response in that specific situation, and optimizing the clinically relevant performance of that therapy.

The term “average polydispersity” (PDI), as used herein, refers to a measure of the distribution of molecular size in a mixture, e.g., as determined by a chromatographic method, such as gel permeation chromatography (See, e.g., Helmut, D. Gel Chromatography, Gel Filtration, Gel Permeation, Molecular Sieves: A Laboratory Handbook; Springer-Verlag, 1969) or size exclusion chromatography (See, e.g., Trathnigg, B. Determination of MWD and Chemical Composition of Polymers by Chromatographic Techniques. Prog. Polym. Sci. 1995, 20, 615-650.), or through dynamic light scattering (See, e.g., Berne, B. J.; Pecora, R. Dynamic Light Scattering. Courier Dover Publications (2000)).

The disclosure is not intended to be limited in any manner by the above exemplary listing of substituents. Additional terms may be defined in other sections of this disclosure.

### DETAILED DESCRIPTION OF CERTAIN EMBODIMENTS

Before the disclosed systems, compositions, methods, reagents, and kits are described in more detail, it should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

The present disclosure relates to the development of a new class of polyesters, so called amino-polyesters (APEs), having ionizable amines (e.g., being positively charged at pH<7). The APEs disclosed herein are useful for the delivery of a variety of agents, described herein, to cells and to subjects. The APEs can be synthesized via ring opening polymerization (ROP) of various cyclic monomers (e.g., lactones and lactides of Formula (Ic)) using tertiary amine-containing nucleophiles (e.g., amines, alcohols, or thiols) as initiators. One step synthesis, utilizing so called “living polymerization,” enables the generation of large libraries of polymers and allows control of the number of the monomer units (q) by varying the stoichiometry between the initiator and the cyclic monomer resulting in monodispersed polymers (with D values close to 1). APEs for Formula (II) can be easily functionalized by reacting the terminal hydroxyl group to provide, e.g., compounds of Formula (III).

### Compounds

Compounds of Formulae (I), (II), and (III) are provided.

In one aspect, provided herein is a compound of Formula (I):

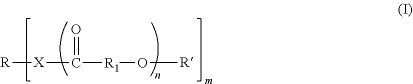

or a pharmaceutically acceptable salt thereof, wherein:

X is O, S, or NR₄;

R is optionally substituted heteroaliphatic, optionally substituted heterocyclyl, or a combination thereof, wherein R comprises one or more amine moieties;

each R₁ independently is optionally substituted aliphatic; optionally substituted carbocyclyl; optionally substituted heteroaliphatic; or optionally substituted heterocyclyl;

R′ is hydrogen, or a group of Formula (Ia):

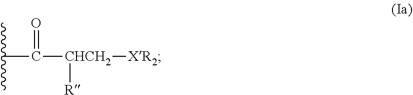

each R″ independently is hydrogen, optionally substituted aliphatic, or optionally substituted heteroaliphatic;

each R₂ independently is hydrogen, optionally substituted aliphatic, optionally substituted heteroaliphatic, optionally substituted carbocyclyl, optionally substituted heterocyclyl, optionally substituted aryl, or optionally substituted heteroaryl;

each X′ independently is O, S, or NR₃;

each R₃ is hydrogen, optionally substituted, aliphatic; optionally substituted carbocyclyl; optionally substituted heteroaliphatic; optionally substituted hetercyclyl; optionally substituted aryl; or optionally substituted heteroaryl; or R₂ and R₃ are combined to form an optionally substituted heterocyclyl;

each R₄ independently is optionally substituted, aliphatic; optionally substituted carbocyclyl; optionally substituted heteroaliphatic; optionally substituted hetercyclyl; optionally substituted aryl; or optionally substituted heteroaryl;

each n independently is an integer between 1 and 20, inclusive; and

m is an integer between 1 and 10, inclusive.

In certain embodiments, said one or more amine moieties of R are all tertiary amines.

In certain embodiments, the compound of Formula (I) is of Formula (II):

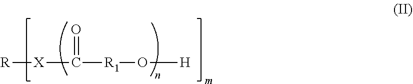

or a pharmaceutically acceptable salt thereof.

In certain embodiments, the compound of Formula (I) is of Formula (III):

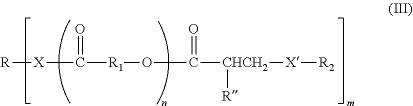

or a pharmaceutically acceptable salt thereof.

In certain embodiments, the compound of Formula (III) is of Formula (IIIa):

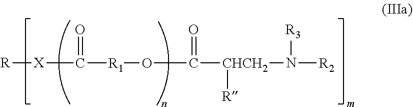

or a pharmaceutically acceptable salt thereof.

In certain embodiments, the compound of Formula (III) is of Formula (IIIb)

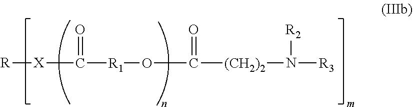

or a pharmaceutically acceptable salt thereof.

In certain embodiments, the compounds of Formulae (I), (II), (III), (IIIa), and (IIIb), and pharmaceutically acceptable salts thereof, have substitutents as defined below.

### Variable R

R is a mono- or poly-valent radical, having m (i.e., 1-10) valencies. R is optionally substituted heteroaliphatic, optionally substituted heterocyclyl, or a combination thereof. R comprises one or more amine moieties. In certain embodiments, all amine moieties of R are tertiary. In certain embodiments, R comprises substituted one or more alkylamine moiety In certain embodiments, R comprises substituted polyalkylamine (e.g., polyethylamine) moieties. In certain embodiments, R comprises substituted piperazine moieties. In certain embodiments, R comprises substituted triazine moieties. In certain embodiments, R comprises a combination of moieties selected from substituted alkylamine, substituted polyalkylamine (e.g., polyethylamine), substituted piperazine, and substituted triazine.

In certain embodiments, R is selected from the following:

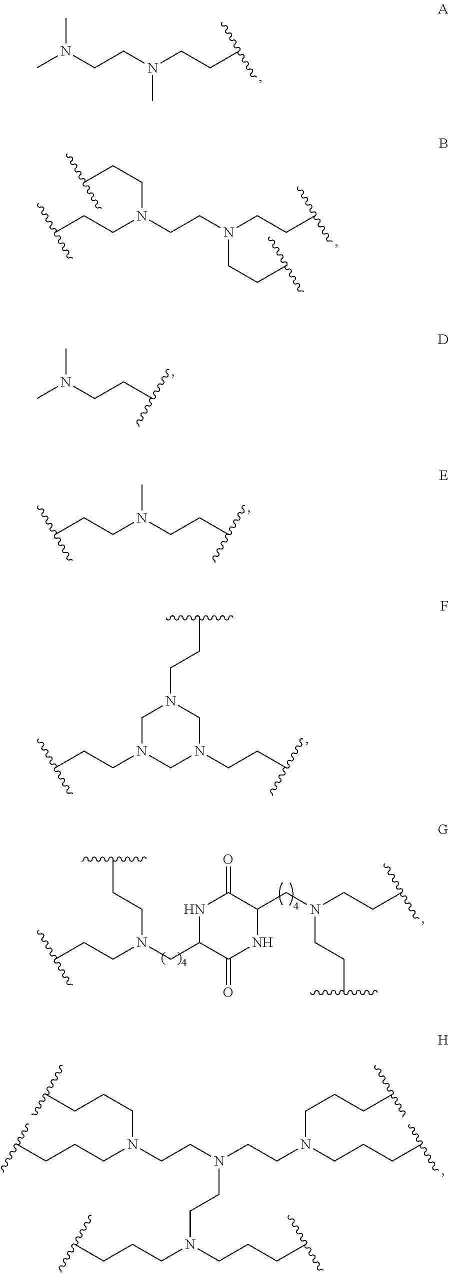

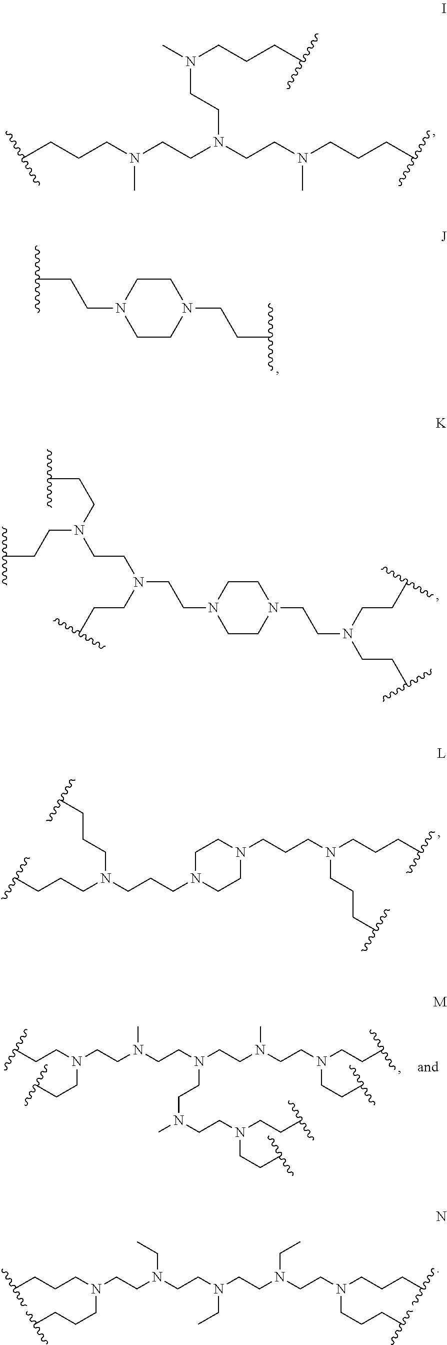 [SMILES: *CCCN(C)CCN(CCN(C)CCC*)CCN(C)CCC* ; *CCN1CCN(CC*)CC1 ; *CCN(CC*)CCN(CC*)CCN1CCN(CCN(CC*)CC*)CC1 ; *CCCN(CCC*)CCCN1CCN(CCCN(CCC*)CCC*)CC1 ; *CCN(CC*)CCN(C)CCN(CCN(C)CCN(CC*)CC*)CCN(C)CCN(CC*)CC* ; *CCCN(CCC*)CCN(CC)CCN(CC)CCN(CC)CCN(CCC*)CCC*]

### Variable R₁

R₁ is a divalent radical. Each R₁ independently is optionally substituted aliphatic; optionally substituted carbocyclylene; optionally substituted heteroaliphatic; or optionally substituted heterocyclylene. In certain embodiments, each R₁ is substituted aliphatic. In certain embodiments, each R₁ is unsubstituted aliphatic. In certain embodiments, each R₁ is substituted C₁-C₆ alkylene. In certain embodiments, each R₁ is unsubstituted C₁-C₆ alkylene.

In certain embodiments, each R₁ is substituted heteroaliphatic. In certain embodiments, each R₁ is unsubstituted heteroaliphatic. In certain embodiments, each R₁ is substituted C₁-C₆ heteroalkylene. In certain embodiments, each R₁ is unsubstituted C₁-C₆ heteroalkylene.

In certain embodiments, each R₁ is substituted carbocyclylene. In certain embodiments, each R₁ is unsubstituted carbocyclylene. In certain embodiments, each R₁ is substituted heterocyclylene. In certain embodiments, each R₁ is unsubstituted heterocyclylene.

In certain embodiments, R₁ is a C₂-C₂₀ aliphatic chain, or a C₂-C₂₀ heteroaliphatic chain, wherein R₁ is substituted one or more times with a group selected from: halogen, cyano, —NHR″′, —NR′″₂, —OR″′, —OC(O)R″—C(O)R′″, —C(O₂)R′″, SR′″, —SC(O)R′″, and —C(O)SR″′; wherein R′″ is hydrogen, a nitrogen protecting group, an oxygen protecting group, a sulfur protecting group, or optionally substituted aliphatic.

In certain embodiments, R₁ is of the formula:

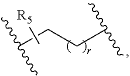

wherein R₅ is optionally substituted aliphatic or optionally substituted heteroaliphatic; and r is 1-19. In the formula above, and related formulae herein, R₅ may be a substituent on any carbon atom, including carbon atoms within parentheses.

In certain embodiments, R₅ is optionally substituted C₁-C₂₀ alkyl. In certain embodiments, R₅ is optionally substituted C₁-C₂₀ heteroalkyl.

In certain embodiments of R₁, r is 1-19. In certain embodiments of R₁, r is 2-10. In certain embodiments of R₁, r is 4-10. In certain embodiments of R₁, r is 6-10.

In certain embodiments, R₁ is selected from the formulae:

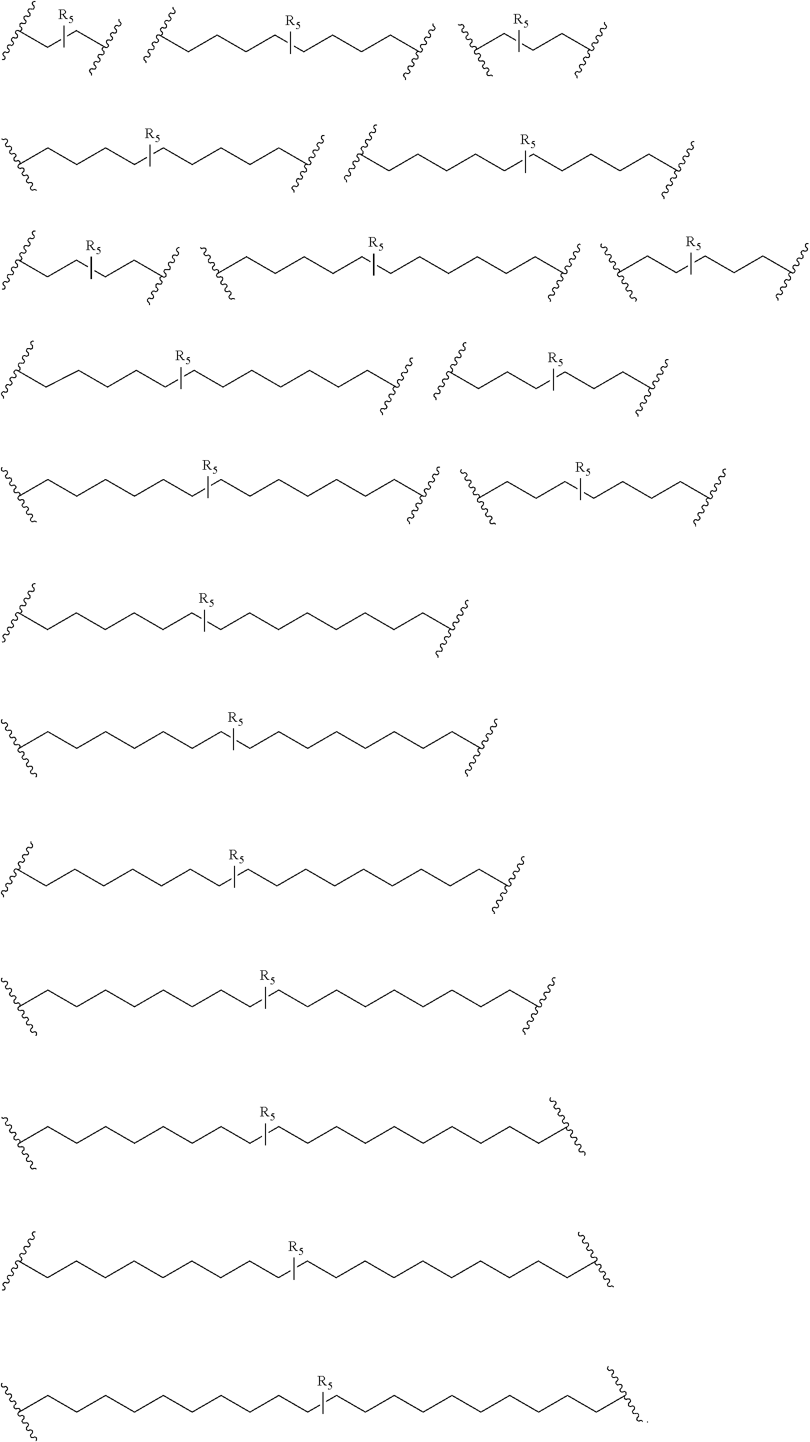

In certain embodiments, R₁ has a structure selected from (CL), (DD) and (TD):

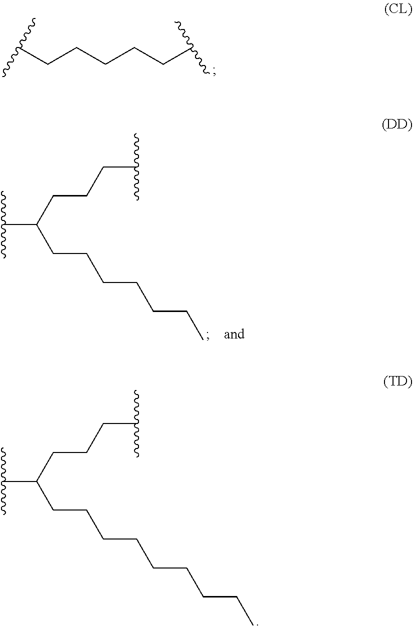 [SMILES: *CCCCC* ; *CCCC(*)CCCCCCC ; *CCCC(*)CCCCCCCCC]

### Variable R₂

R₂ is a monovalent radical. In certain embodiments of the compounds described herein, each instance of R₂ is the same. In certain embodiments, each instance of R₂ is not the same. In certain embodiments, R₂ independently is hydrogen, optionally substituted, cyclic or acyclic aliphatic, optionally substituted, cyclic or acyclic heteroaliphatic, optionally substituted aryl, or optionally substituted heteroaryl. In certain embodiments, each R₂ is hydrogen. In certain embodiments, each R₂ independently is substituted acyclic aliphatic. In certain embodiments, each R₂ independently is unsubstituted acyclic aliphatic. In certain embodiments, each R₂ independently is substituted cyclic aliphatic. In certain embodiments, each R₂ independently is unsubstituted cyclic aliphatic. In certain embodiments, each R₂ independently is substituted acyclic heteroaliphatic. In certain embodiments, each R₂ independently is unsubstituted acyclic heteroaliphatic. In certain embodiments, each R₂ independently is substituted cyclic heteroaliphatic. In certain embodiments, each R₂ independently is unsubstituted cyclic heteroaliphatic. In certain embodiments, each R₂ independently is substituted aryl. In certain embodiments, each R₂ independently is unsubstituted aryl. In certain embodiments, each R₂ independently is substituted heteroaryl. In certain embodiments, each R₂ independently is unsubstituted heteroaryl.

In certain embodiments, each R₂ independently is optionally substituted, cyclic or acyclic aliphatic, or optionally substituted, cyclic or acyclic heteroaliphatic. In certain embodiments, each R₂ independently is:

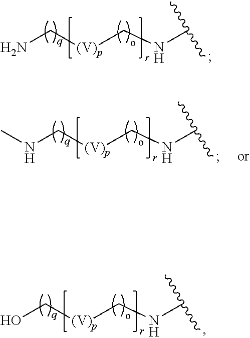

wherein: o, p, q, and r are each independently an integer between 0 and 20, inclusive; each instance of V is independently —O—, —S—, —NH—, —NR_{V}—, or C(R_{V})₂, wherein each instance of R_{V} is independently hydrogen, halogen, hydroxyl, C₁₋₆aliphatic, C₁₋₆heteroaliphatic, C₁₋₆alkoxy, amino, C₁₋₆alkylamino, di(C¹⁻⁶alkyl)amino, aryl, heteroaryl, thiol, alkylthioxy, or acyl. In certain embodiments, each R₂ independently is

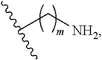

wherein m is an integer between 1 and 20, inclusive. In certain embodiments, each R₂ independently is

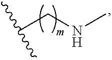

wherein m is an integer between 1 and 20, inclusive. In certain embodiments, each R₂ independently is

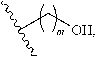

wherein m is an integer between 1 and 20, inclusive. In certain embodiments, each R₂ independently is

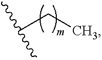

wherein m is an integer between 1 and 20, inclusive. In certain embodiments, each R₂ independently is

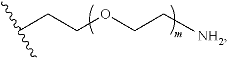

wherein m is an integer between 1 and 20, inclusive. In certain embodiments, each R₂ independently is

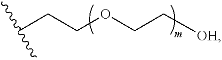

wherein m is an integer between 1 and 20, inclusive. In certain embodiments, each R₂ independently is

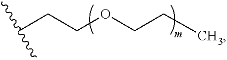

wherein m is an integer between 1 and 20, inclusive.

In certain embodiments, each R₂ and independently is selected from the group consisting of:

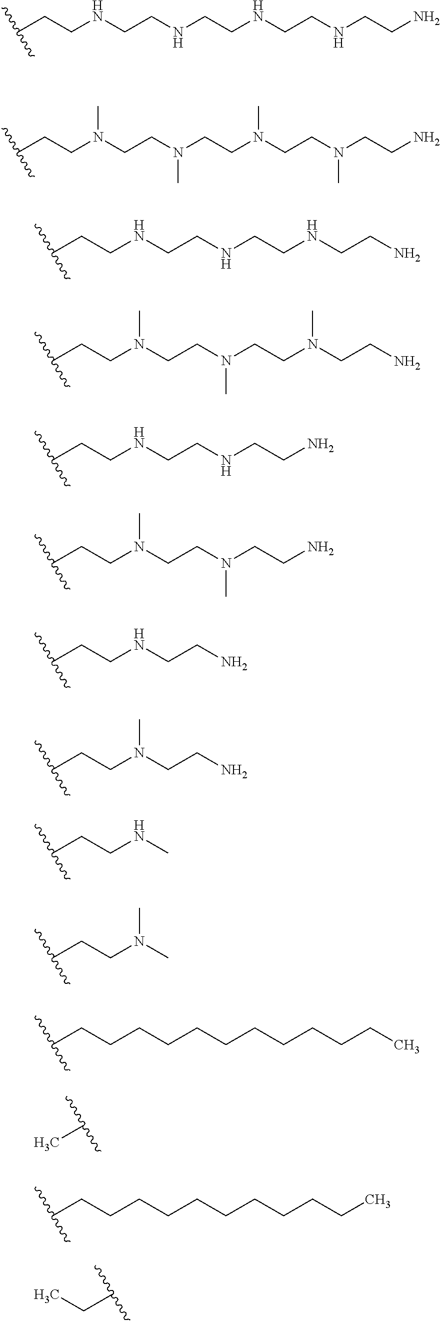

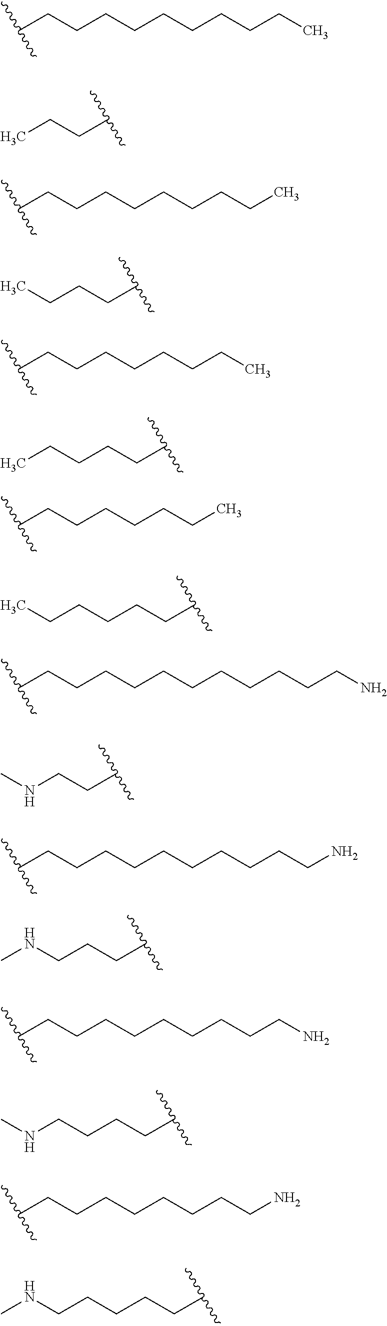

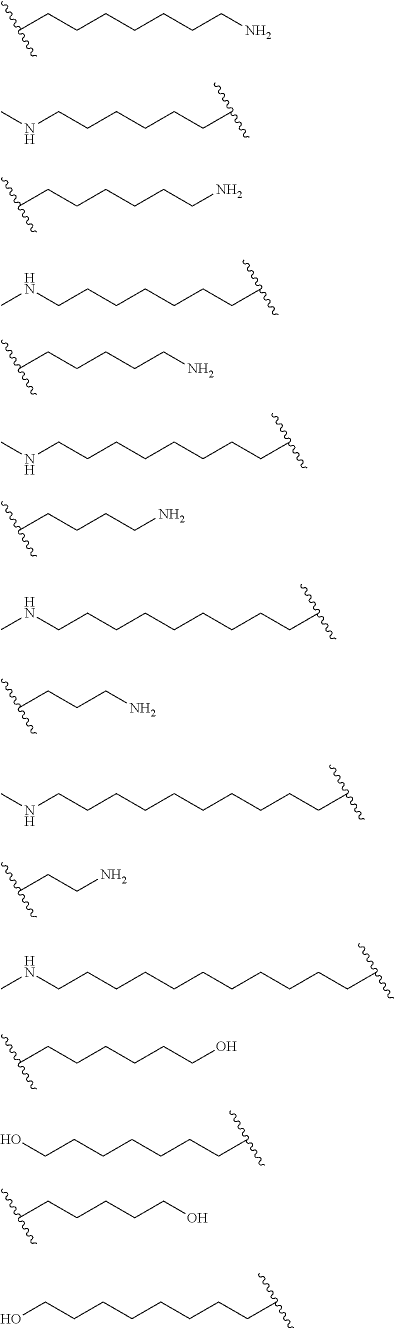

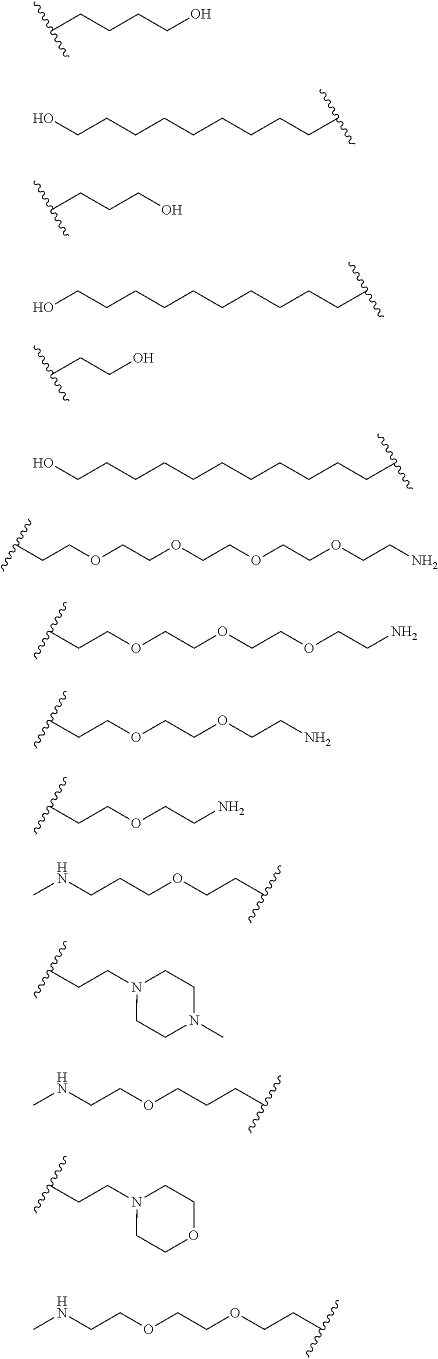

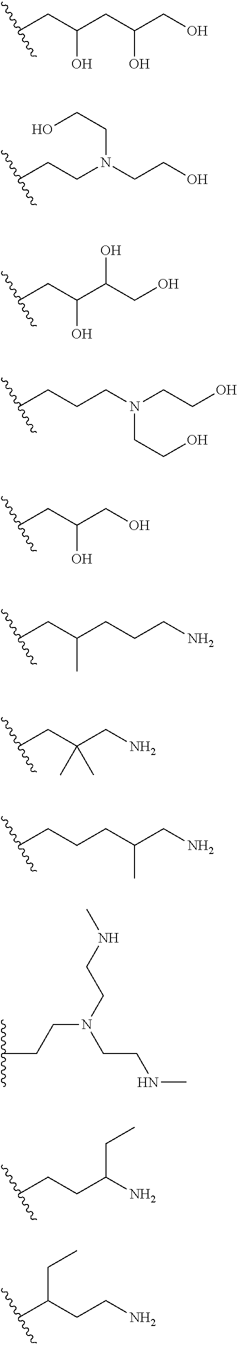

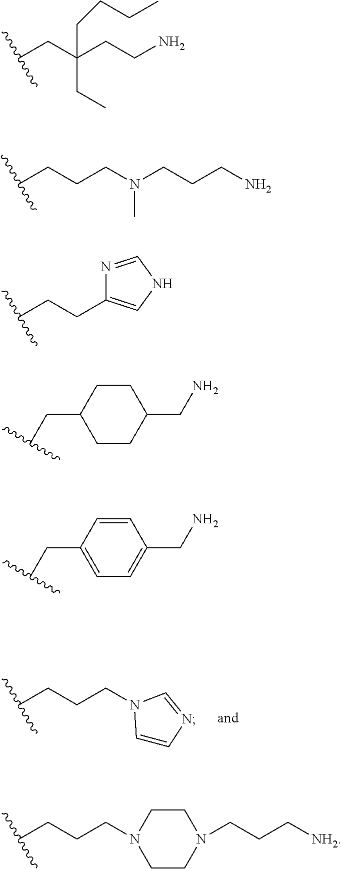

In certain embodiments, each R₂ independently is selected from:

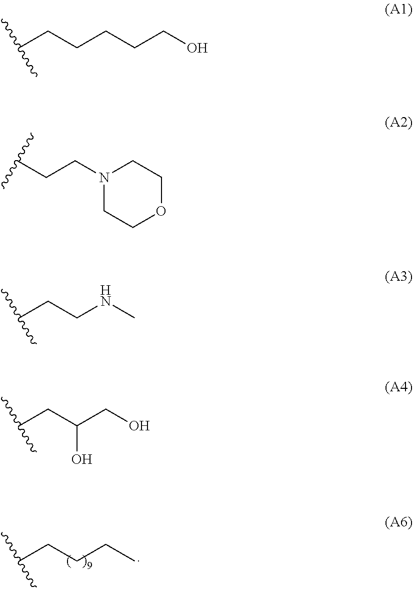

### Variable R₃

Variable R₃ is a monovalent radical. In certain embodiments, R₃ is selected from the possibilities for R₂, listed above. In certain embodiments, each R₃ independently is hydrogen, optionally substituted, aliphatic; optionally substituted carbocyclyl; optionally substituted heteroaliphatic; optionally substituted hetercyclyl; optionally substituted aryl; or optionally substituted heteroaryl. In certain embodiments, each R₃ is hydrogen. In certain embodiments, each R₃ independently is substituted aliphatic. In certain embodiments, each R₃ is independently unsubstituted aliphatic. In certain embodiments, each R₃ is independently substituted or unsubstituted C₁₋₆ alkyl. In certain embodiments, each R₃ is hydrogen or unsubstituted methyl. In certain embodiments, each R₃ is unsubstituted methyl.

In certain embodiments, R₂ and R₃ are combined to form a ring, e.g., a 4-, 5-, 6-, or 7-membered optionally substituted, saturated, unsaturated, or partially-unsaturated ring. In certain particular embodiments, R₂ and R₃ are combined to form an optionally substituted azetidine, pyrroline, piperidine, morpholine, piperazine, pyrazole, or imidazole moiety.

### Variable R₄

In certain embodiments, each R₄ independently is hydrogen, optionally substituted, aliphatic; optionally substituted carbocyclyl; optionally substituted heteroaliphatic; optionally substituted hetercyclyl; optionally substituted aryl; or optionally substituted heteroaryl. In certain embodiments, each R₄ is hydrogen. In certain embodiments, each R₄ independently is substituted aliphatic. In certain embodiments, each R₄ is independently unsubstituted aliphatic. In certain embodiments, each R₄ is independently substituted or unsubstituted C₁₋₆ alkyl. In certain embodiments, each R₄ is hydrogen or unsubstituted methyl. In certain embodiments, each R₄ is unsubstituted methyl.

### Variable R₅

R₅ is a monovalent radical. In certain embodiments, R₅ is selected from the group consisting of alkyl, heteroalkyl, alkenyl, alkynyl, haloalkyl, alkoxy, thioalkoxy, amino, alkylamino, dialkylamino, trialkylamine, acylamino, cyano, hydroxy, halo, mercapto, nitro, carboxyaldehyde, carboxy, alkoxycarbonyl, and carboxamide, wherein said alkyl, heteroalkyl, alkenyl, and alkynyl are optionally substituted, cyclic or acyclic, and branched or linear.

In certain embodiments, R₅ is substituted C₁-C₂₀ alkyl. In certain embodiments, R₅ is unsubstituted C₁-C₂₀ alkyl. In certain embodiments, R₅ is optionally substituted C₁-C₂₀ heteroalkyl. In certain embodiments, R₅ is optionally unsubstituted C₁-C₂₀ heteroalkyl.

### Variable R′

R′ is a monovalent radical selected from hydrogen and a group of Formula (Ia):

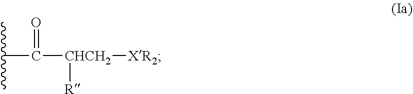

wherein X′, R″ and R₂ are as defined herein. In certain particular embodiments, R′ is hydrogen. In certain particular embodiments, R′ is of Formula (Ia).

In certain embodiments, the radical of Formula (Ia) is selected from the formulae:

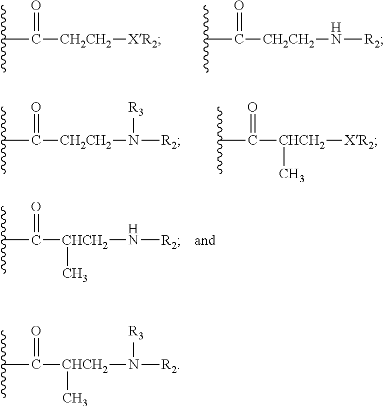

### Variable R″

Each R″ is a monovalent radical selected from hydrogen, halogen, optionally substituted aliphatic, and optionally substituted heteroaliphatic. In certain embodiments, each R″ is hydrogen. In certain embodiments, each R″ is halogen. In certain embodiments, each R″ is substituted aliphatic. In certain embodiments, each R″ is substituted C₁-C₆ alkyl. In certain embodiments, each R″ is unsubstituted aliphatic. In certain embodiments, each R″ is unsubstituted C₁-C₆ alkyl. In certain particular embodiments, R″ is methyl.

In certain embodiments, each R″ is substituted heteroaliphatic. In certain embodiments, each R″ is substituted C₁-C₆ heteroalkyl. In certain embodiments, each R″ is unsubstituted heteroaliphatic. In certain embodiments, each R″ is unsubstituted C₁-C₆ heteroalkyl.

### Variable m

In certain embodiments, m is an integer between 1 and 10, inclusive. In certain embodiments, m is an integer between 1 and 6, inclusive. In certain embodiments, m is 1, 2, 3, 4, 5, 6, 7, 8, 9, or 10.

### Variable n

In certain embodiments, n is an integer between 1 and 20, inclusive. In certain embodiments, n is an integer between 1 and 10, inclusive. In certain embodiments, n is an integer between 3 and 10, inclusive. In certain embodiments, n is 1, 2, 3, 4, 5, 6, 7, 8, 9, or 10.

### Specific Embodiments

In certain embodiments of Formula (II), R, X, R₁, n, and m are as defined herein. In certain embodiments, R, X, R₁, n, and m are as follows:

| (II) | R | X | R₁ | n | m |
|---|---|---|---|---|---|
| IDD3 | 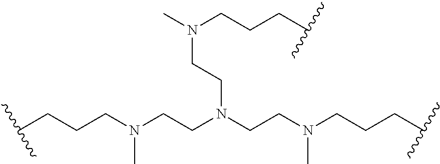 [SMILES: *CCCN(C)CCN(CCN(C)CCC*)CCN(C)CCC*] | O | 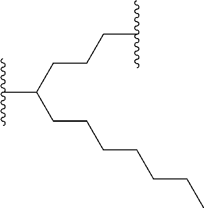 [SMILES: *CCCC(*)CCCCCCC] | 3 | 3 |
| ITD3 | 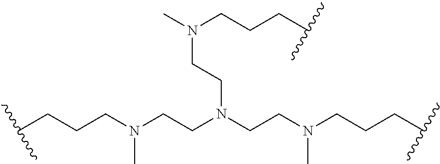 [SMILES: *CCCN(C)CCN(CCN(C)CCC*)CCN(C)CCC*] | O | 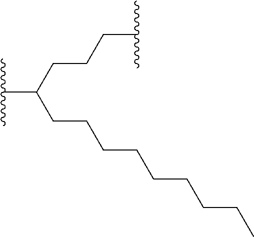 [SMILES: *CCCC(*)CCCCCCCCC] | 3 | 3 |
| ATD3 | 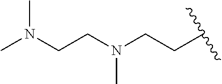 [SMILES: *CCN(C)CCN(C)C] | O | 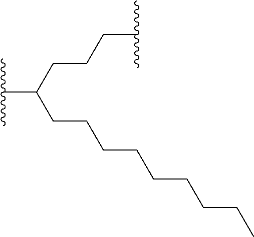 [SMILES: *CCCC(*)CCCCCCCCC] | 3 | 1 |
| BDD3 | 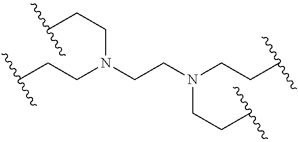 [SMILES: *CCN(CC*)CCN(CC*)CC*] | O | 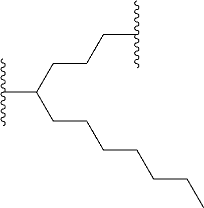 [SMILES: *CCCC(*)CCCCCCC] | 3 | 4 |
| ADD5 | 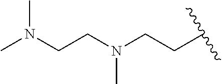 [SMILES: *CCN(C)CCN(C)C] | O | 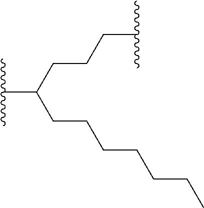 [SMILES: *CCCC(*)CCCCCCC] | 5 | 1 |
| BTD3 | 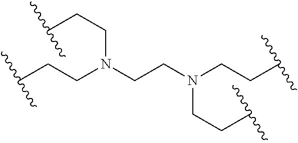 [SMILES: *CCN(CC*)CCN(CC*)CC*] | O | 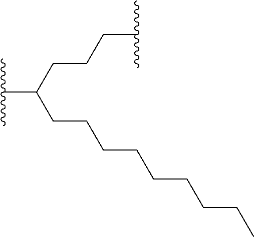 [SMILES: *CCCC(*)CCCCCCCCC] | 3 | 4 |
| HTD3 | 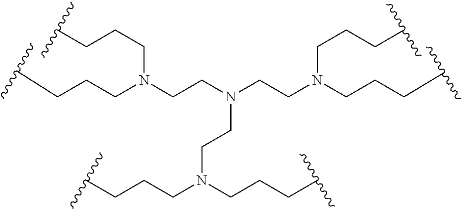 [SMILES: *CCCN(CCC*)CCN(CCN(CCC*)CCC*)CCN(CCC*)CCC*] | O | 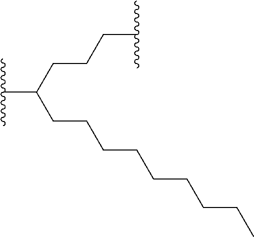 [SMILES: *CCCC(*)CCCCCCCCC] | 3 | 6 |
| BTD5 | 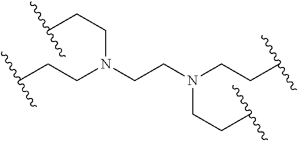 [SMILES: *CCN(CC*)CCN(CC*)CC*] | O | 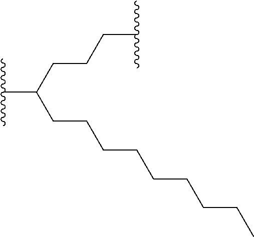 [SMILES: *CCCC(*)CCCCCCCCC] | 5 | 4 |
| BDD5 | 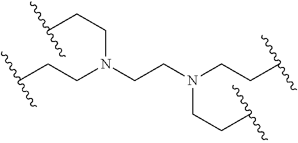 [SMILES: *CCN(CC*)CCN(CC*)CC*] | O | 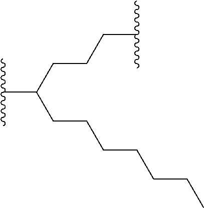 [SMILES: *CCCC(*)CCCCCCC] | 5 | 4 |
| DTD5 | 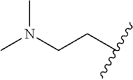 [SMILES: *CCN(C)C] | O | 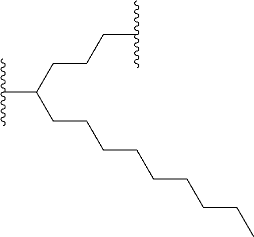 [SMILES: *CCCC(*)CCCCCCCCC] | 5 | 1 |
| ETD5 | 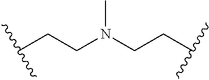 [SMILES: *CCN(C)CC*] | O | 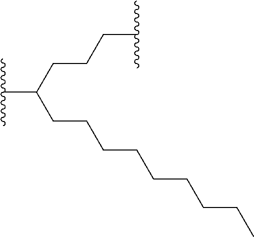 [SMILES: *CCCC(*)CCCCCCCCC] | 5 | 2 |
| FDD5 | 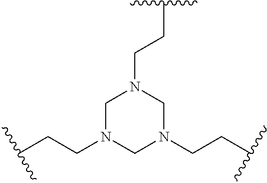 [SMILES: *CCN1CN(CC*)CN(CC*)C1] | O | 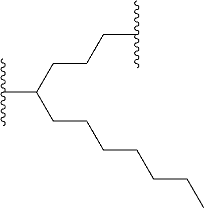 [SMILES: *CCCC(*)CCCCCCC] | 5 | 3 |

In certain embodiments of Formula (III), R, X, R₁, R₂, R₃, n, and m are as defined herein. In certain embodiments, R, X, R₁, n, and m are as follows:

| (III) | R | X | R₁ | R₂ | R₃ | n | m |
|---|---|---|---|---|---|---|---|
| BDD3-Ac1 | 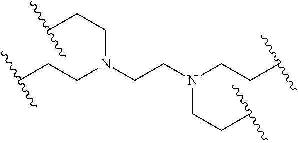 [SMILES: *CCN(CC*)CCN(CC*)CC*] | O | 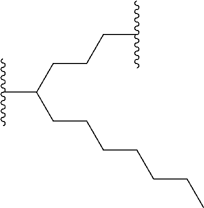 [SMILES: *CCCC(*)CCCCCCC] | H | 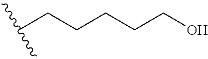 [SMILES: *CCCCCO] | 3 | 4 |
| BDD3-Ac2 | 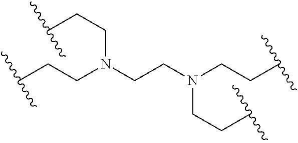 [SMILES: *CCN(CC*)CCN(CC*)CC*] | O | 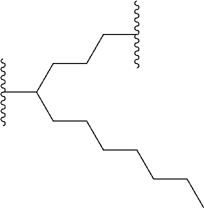 [SMILES: *CCCC(*)CCCCCCC] | H | 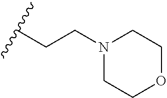 [SMILES: *CCN1CCOCC1] | 3 | 4 |
| BDD3-Ac3 | 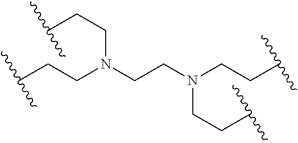 [SMILES: *CCN(CC*)CCN(CC*)CC*] | O | 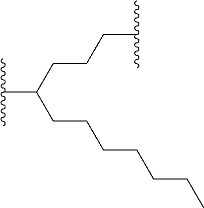 [SMILES: *CCCC(*)CCCCCCC] | H | 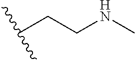 [SMILES: *CCNC] | 3 | 4 |
| BDD3-Ac4 | 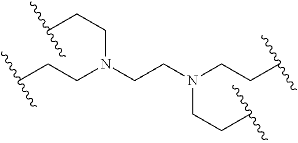 [SMILES: *CCN(CC*)CCN(CC*)CC*] | O | 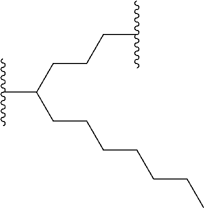 [SMILES: *CCCC(*)CCCCCCC] | H | 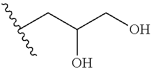 [SMILES: *CC(O)CO] | 3 | 4 |
| BDD3-Ac6 | 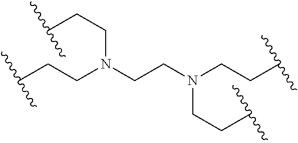 [SMILES: *CCN(CC*)CCN(CC*)CC*] | O | 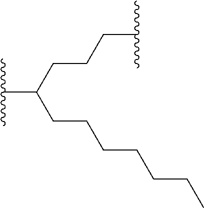 [SMILES: *CCCC(*)CCCCCCC] | H | 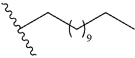 | 3 | 4 |

### Physical Properties

Exemplary APE compounds may be described in terms of properties including, weight average molecular weight (M_{w}), number average molecular weight (Mₙ), average hydrodynamic diameter (D_{H}), and polydispersity (Ð).

Mₙ is the average molecular weight of all the polymer chains in a sample, and is defined by the formula:

$$Mn = \frac{\sum {N}_{i} {M}_{i}}{\sum {N}_{i}}$$

where Mᵢ is the molecular weight of a chain and Nᵢ is the number of chains having that molecular weight. Mₙ can be predicted by polymerization mechanisms and is measured by methods that determine the number of molecules in a sample of a given weight; for example, colligative methods such as end-group assay.

In certain embodiments, the Mₙ is determined with viscometry via the Mark-Houwink equation, colligative methods (such as vapor pressure osmometry), end-group determination, or proton NMR (Izunobi, J. U., et al., J. Chem. Educ., 2011, 88, 1098-1104).

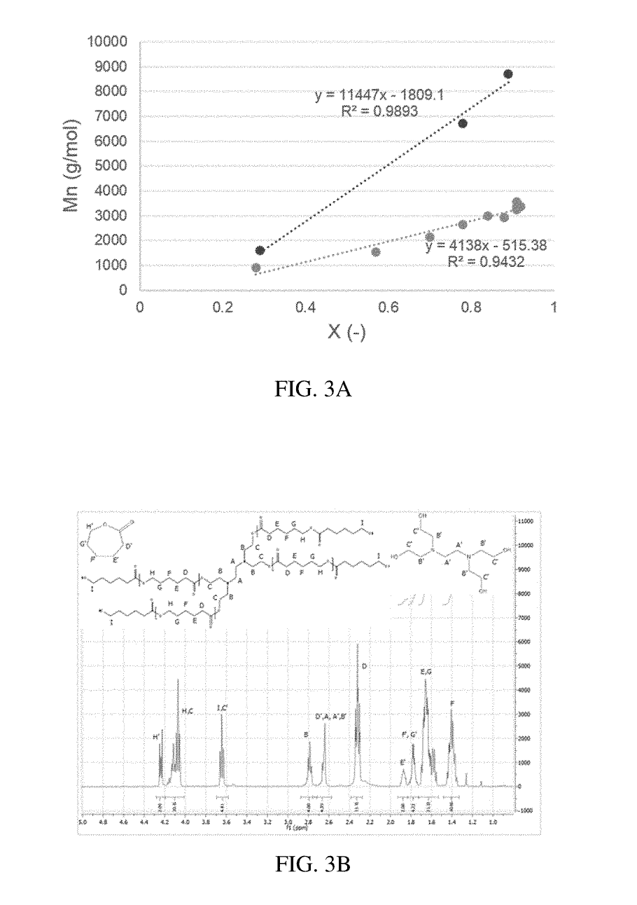

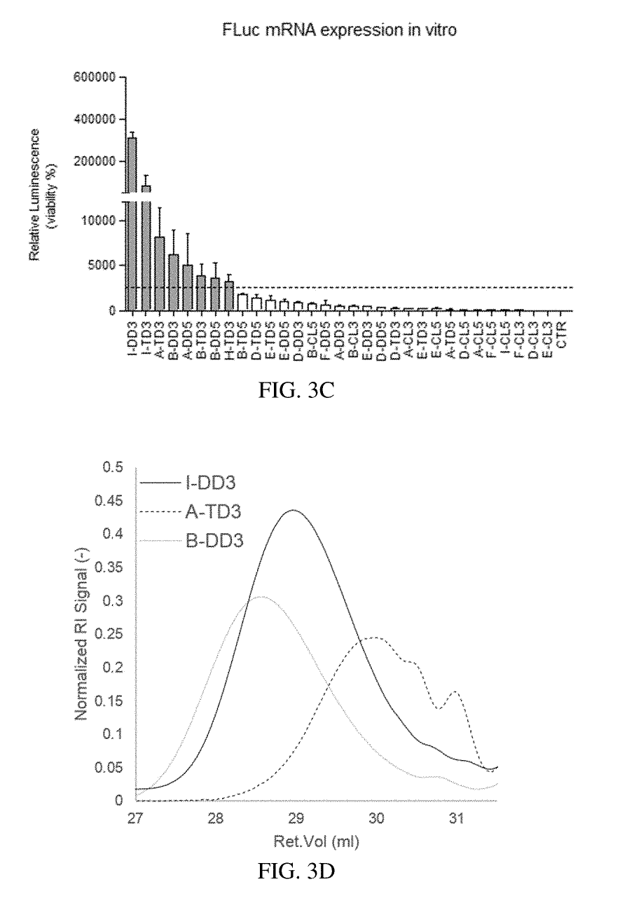

In certain embodiments, Mₙ is about 1000 to about 9000 Da, e.g., as determined by gel permeation chromatography. In some embodiments, Mₙ is about 1000 to about 3500 Da. In some embodiments, Mₙ is about 1500 to about 9000 Da. See, e.g., FIG. 3A.

M_{w} is defined by the formula:

$$Mw = \frac{\sum {N}_{i} {M}_{i}^{2}}{\sum {N}_{i} {M}_{i}}$$

where Mᵢ and Nᵢ are as defined above.

M_{w} is determined by methods that are sensitive to the molecular size, such as light scattering techniques. If M_{w} is quoted for a molecular weight distribution, there is equal weight of molecules on either side of the M_{w} in the distribution.

The weight average molecular mass can be determined by gel permeation chromatography, static light scattering (See, e.g., Zimm, B. H., J. Chem. Phys., 1945, 13, 141), small angle neutron scattering (See, e.g., Jacrot, B., Reports on Progress in Physics, 1976, 39, 911-53), X-ray scattering (See, e.g., Foster, M. D., Critical Reviews in Analytical Chemistry, 2006, 24, 179-241), and sedimentation velocity (See, e.g., Ghirlando, R., Modern Analytical Ultracentrifugation: Methods, 2011, 58, 145-156).

The average hydrodynamic diameter (D_{H}) is measured by dynamic light scattering (DLS). (See, e.g., Chu, B., Annual Review of Physical Chemistry, 1970, 21, 145-174). Polydispersity (Ð) is a measure of the distribution of molecular mass in a given polymer. Polydispersity is calculated by: Ð=M_{w}/Mₙ (See, e.g., Step to, R. F. T., et al., Pure Appl. Chem., 2009, 81, 351-353).

### Methods of Preparation

As detailed in Examples 1 and 2, amino-polyesters (APE) compounds of the present disclosure can be synthesized via ring opening polymerization.

In another aspect, provided herein a method of making a compound of Formula (II), or a salt thereof, comprising acylating a compound of Formula (Ib):

R(X)ₘ (Ib)

or a salt thereof, wherein R, X and m are defined herein, with a compound of Formula (Ic):

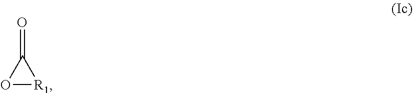

wherein R₁ is defined herein, to obtain a compound of Formula (II), or a salt thereof.

In another aspect, provided herein is a method of making a compound of Formula (III), or a salt thereof, comprising acylating a compound of Formula (II) to obtain a compound of Formula (IIa):

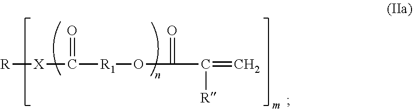

wherein X, R, R₁, and R″ are defined herein, and alkylating a compound of Formula (IIb):

HX′R₂ (IIb)

with the compound of Formula (IIa) to obtain the compound of Formula (III), or a salt thereof.

In certain embodiments, the step of acylating the compound of Formula (Ib) comprises contacting the compound of Formula (Ib) with the compound of Formula (Ic) in the presence of a catalyst. The catalyst may be an organic molecule or an inorganic (e.g., transition metal-containing) molecule. In certain particular embodiments, the catalyst comprises a guanidine moiety. In certain particular embodiments, the catalyst is triazabicyclodecene. In certain particular embodiments, the catalyst is dimethylaminopyridine.

In certain embodiments, the step of acylating a compound of Formula (II) is conducted at temperature in the range of about −78° C. to about 100° C. In certain particular embodiments, the temperature is less than about 25° C. In certain particular embodiments, the temperature about 0° C.

In certain embodiments, the step of acylating a compound of Formula (II) comprises reacting the compound of Formula (II) with an acylating agent of Formula (IIc):

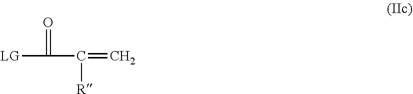

wherein LG is a leaving group as defined herein. In certain particular embodiments, LG is Cl. In certain particular embodiments, LG is of the formula:

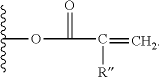

### Compositions and Kits

The present disclosure provides compositions (e.g., pharmaceutical compositions) comprising a polymer described herein, and an excipient (e.g., pharmaceutically acceptable excipient). In certain embodiments, the composition is a pharmaceutical composition. In certain embodiments, the composition is a cosmetic composition. In certain embodiments, the composition is a nutraceutical composition. In certain embodiments, the composition is a composition with a non-medical application. In certain embodiments, the excipient is a pharmaceutically acceptable excipient.

In certain embodiments, an APE compound as described herein (e.g., a compound of Formulae (I), (II), or (III)) is in the form of a composition having a polydispersity of about 1 to about 2 (e.g., 1.0-2.0).

Compositions described herein can be prepared by any method known in the art. In general, such preparatory methods include bringing the polymer described herein into association with one or more excipients, and may include one or more agents and then, if necessary and/or desirable, shaping, and/or packaging the product into a desired single- or multi-dose unit. In certain embodiments, the agent and the polymer of the composition are not covalently attached.

In certain embodiments, the composition is in the form of a particle. In certain embodiments, the particle is a nanoparticle or a microparticle. In certain embodiments, the particle is a micelle, liposome, polyplex or lipoplex. In certain embodiments, the particle encapsulates an agent, as described herein. In certain embodiments, the particle facilitates delivery of the agent to a cell. In certain embodiments, the particle facilitates delivery of the agent to a subject, e.g., a human.

Nanoparticles and nanoparticle formulations are described herein. In certain embodiments, the nanoparticle comprises one or more additional lipids. In certain particular embodiments, the nanoparticle comprises a PEG-lipid. As described herein, polycaprolactone (PCL)-based APE nanoparticles can be prepared using a pre-mixing protocol or a direct-mixing protocol.

Compositions can be prepared, packaged, and/or sold in bulk, as a single unit dose, and/or as a plurality of single unit doses. A “unit dose” is a discrete amount of the composition comprising a predetermined amount of the agent. The amount of the agent is generally equal to the dosage of the agent which would be administered to a subject and/or a convenient fraction of such a dosage, such as one-half or one-third of such a dosage.

Relative amounts of the polymer, excipient, agent, and/or any additional ingredients in a composition described herein will vary, depending upon the identity, size, and/or condition of the subject treated and further depending upon the route by which the composition is to be administered. The composition may comprise between 0.1% and 100% (w/w) agent.

Excipients and accessory ingredients used in the manufacture of provided compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter, PEGylated lipids, phospholipids, suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SEES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In certain embodiments, the compositions, further comprise an agent, and are useful for delivering said agent (e.g., to a subject or cell). In certain embodiments, the compositions are pharmaceutical compositions which are useful for treating a disease in a subject in need thereof. In certain embodiments, the pharmaceutical compositions are useful for preventing a disease in a subject. In certain embodiments, the pharmaceutical compositions are useful for diagnosing a disease in a subject.

A composition as described herein may further comprise, or can be administered in combination with, one or more additional agents. In certain embodiments, the agent is a small organic molecule, inorganic molecule, nucleic acid, protein, peptide, or polynucleotide. In certain embodiments, the agent is a pharmaceutical agent (e.g., therapeutically and/or prophylactically active agent). Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, polynucleotides, lipids, hormones, vitamins, vaccines, immunological agents, and cells or other biological materials.

In certain embodiments, the agent is a polynucleotide. In certain embodiments, the polynucleotide is DNA. In certain embodiments, the polynucleotide is RNA. In certain embodiments, the polynucleotide carries out RNA interference. The RNA is selected from the group consisting of double-stranded RNA (dsRNA), small interfering RNA (siRNA), short hairpin (shRNA), microRNA (miRNA), messenger RNA (mRNA), antisense RNA, transfer RNA (tRNA), small nuclear RNA (snRNA), and ribosomal RNA (rRNA). In certain embodiments, the RNA is dsRNA. In certain embodiments, the RNA is siRNA. In certain embodiments, the RNA is shRNA. In certain embodiments, the RNA is miRNA. In certain embodiments, the RNA is mRNA. In certain embodiments, the RNA is antisense RNA.

In certain embodiments, the agent described herein is provided in an effective amount in the composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, infectious disease, inflammatory disease, autoimmune disease, gastrointestinal disease, liver disease, lung disease, kidney disease, spleen disease, familial amyloid neuropathies, painful condition, psychiatric disorder, or metabolic disorder) in a subject in need thereof.

In certain embodiments, the cell is in vitro. In certain embodiments, the cell is in vivo.

Compositions may be formulated into liquid dosage forms for oral and parenteral administration include pharmaceutically acceptable emulsions, microemulsions, solutions, suspensions, syrups and elixirs. In addition to the agents, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the particles described herein are mixed with solubilizing agents, such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids, such as oleic acid, are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an agent, it is often desirable to slow the absorption of the drug from subcutaneous or intramuscular injection. This can be accomplished by the use of a liquid suspension of crystalline or amorphous material with poor water solubility. The rate of absorption of the agent then depends upon its rate of dissolution, which, in turn, may depend upon crystal size and crystalline form.

Alternatively, delayed absorption of a parenterally administered drug form may be accomplished by dissolving or suspending the agent in an oil vehicle.

Compositions for rectal or vaginal administration are typically suppositories which can be prepared by mixing the particles described herein with suitable non-irritating excipients or carriers such as cocoa butter, polyethylene glycol, or a suppository wax which are solid at ambient temperature but liquid at body temperature and therefore melt in the rectum or vaginal cavity and release the agent.

Compositions may be formulated into solid dosage forms for oral administration include capsules, tablets, pills, powders, and granules. In such solid dosage forms, the agent is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the agent(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The agent can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the agent can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the agent(s) only, or preferentially, in a certain part of the intestinal tract, optionally, in a delayed manner. Examples of encapsulating agents which can be used include polymeric substances and waxes.

Dosage forms for topical and/or transdermal administration of a composition described herein may include ointments, pastes, creams, lotions, gels, powders, solutions, sprays, inhalants, and/or patches. Generally, the agent is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an agent to the body. Such dosage forms can be prepared, for example, by dissolving and/or dispensing the agent in the proper medium. Alternatively or additionally, the rate can be controlled by either providing a rate controlling membrane and/or by dispersing the agent in a polymer matrix and/or gel.

Suitable devices for use in delivering intradermal compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the polymer in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) agent, although the concentration of the agent can be as high as the solubility limit of the agent in the solvent. Formulations for topical administration may further comprise one or more of the additional ingredients described herein.

A composition described herein can be prepared, packaged, and/or sold in a formulation suitable for pulmonary administration via the buccal cavity. Such a formulation may comprise dry particles which comprise the agent and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the agent dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the agent may constitute 0.1 to 20% (w/w) of the composition. The propellant may further comprise additional ingredients such as a liquid non-ionic and/or solid anionic surfactant and/or a solid diluent (which may have a particle size of the same order as particles comprising the agent).

Compositions described herein formulated for pulmonary delivery may provide the agent in the form of droplets of a solution and/or suspension. Such formulations can be prepared, packaged, and/or sold as aqueous and/or dilute alcoholic solutions and/or suspensions, optionally sterile, comprising the agent, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate.

Formulations described herein as being useful for pulmonary delivery are useful for intranasal delivery of a pharmaceutical composition described herein. Another formulation suitable for intranasal administration is a coarse powder comprising the agent and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the agent, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) agent, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the agent. Such powdered, aerosolized, and/or aerosolized formulations, when dispersed, may have an average particle and/or droplet size in the range from about 0.1 to about 200 nanometers, and may further comprise one or more of the additional ingredients described herein.

A composition described herein can be prepared, packaged, and/or sold in a formulation for ophthalmic administration. Such formulations may, for example, be in the form of eye drops including, for example, a 0.1-1.0% (w/w) solution and/or suspension of the agent in an aqueous or oily liquid carrier or excipient. Such drops may further comprise buffering agents, salts, and/or one or more other of the additional ingredients described herein. Other opthalmically-administrable formulations which are useful include those which comprise the agent in microcrystalline form and/or in a liposomal preparation. Ear drops and/or eye drops are also contemplated as being within the scope of this disclosure.

Although the descriptions of compositions provided herein are principally directed to compositions which are suitable for administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and/or perform such modification with ordinary experimentation.

Compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific agent employed; the specific composition employed; the age, body weight, general health, sex, and diet of the subject; the time of administration, route of administration, and rate of excretion of the specific agent employed; the duration of the treatment; drugs used in combination or coincidental with the specific agent employed; and like factors well known in the medical arts.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a polymer described herein. In certain embodiments, a dose described herein includes independently between 1 mg and 3 mg, inclusive, of a polymer described herein. In certain embodiments, a dose described herein includes independently between 3 mg and 10 mg, inclusive, of a polymer described herein. In certain embodiments, a dose described herein includes independently between 10 mg and 30 mg, inclusive, of a polymer described herein. In certain embodiments, a dose described herein includes independently between 30 mg and 100 mg, inclusive, of a polymer described herein.

Dose ranges as described herein provide guidance for the administration of provided compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

The compositions can be administered in combination with additional agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a composition described herein including a polymer described herein and an agent shows a synergistic effect that is absent in a composition including one of the polymer and an agent, but not both.

The composition can be administered concurrently with, prior to, or subsequent to one or more additional agents, which are different from the composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the polymer or composition described herein in a single dose or administered separately in different doses. The particular combination to employ in a regimen will take into account compatibility of the polymer described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the polymers described herein or pharmaceutical compositions can be administered in combination with an anti-cancer therapy including surgery, radiation therapy, transplantation (e.g., stem cell transplantation, bone marrow transplantation), immunotherapy, and chemotherapy.

In some embodiments, the composition is a particle (e.g., a nanoparticle). In some embodiments, the particle is substantially soluble in water (e.g., hydrophilic). In some embodiments, the particle is substantially insoluble in water (e.g., hydrophobic). In some embodiments, the particle is substantially insoluble in water and greater than about 10,000 parts water are required to dissolve 1 part polymer. In one embodiment, the particle is amphiphilic. In one embodiment, the particle comprises a segment that is hydrophobic and a segment that is hydrophilic.

In some embodiments, the percentage of the particles that comprise an agent is between about 1 and about 100% (e.g., about 1%, about 2%, about 3%, about 4%, about 5%, about 10%, about 15%, about 20%, about 30%, about 40%, about 50%, about 60%, about 70%, about 80%, about 90%, or about 100%). In some embodiments, the percentage of the particles that comprise an agent is less than about 50%, e.g., less than about 40%, less than about 35%, less than about 30%, less than about 25%, less than about 20%, less than about 15%, or less than about 10%. In some embodiments, the percentage of the particles that comprise an agent is between about 5% and about 50%, about 5% and about 40%, about 5% and about 30%, about 5% and about 25%, or about 5% and about 20%. In some embodiments, the percentage of the particles that comprise an agent is between about 5% and 90%. In some embodiments, the percentage of the particles that comprise an agent is between about 5% and about 75%. In the some embodiments, the percentage of particles that comprise an agent is between about 5% and about 50%. In the some embodiments, the percentage of the particles that comprise an agent is between about 10% and about 25%.

In some embodiments, the total amount of the agent present in the particle is greater than about 5% (e.g., about 6%, about 7%, about 8%, about 9%, about 10%, about 12%, about 15%, about 20%, about 25%, about 30%, or more) of the total size or weight of the conjugate or particle. In some embodiments, the total amount of the agent present in the conjugate or particle is greater than about 10% (e.g., about 12%, about 15%, about 20%, about 25%, about 30%, or more) of the total size or weight of the conjugate or particle.

Without being bound by theory, the polymers or particles disclosed herein may improve the efficiency of an agent by one or more of increasing the localization and/or release (e.g., preferential release) of the agent to a target cell (e.g., a cancer or a fibrotic cell; a cell associated with a hypoxic environment), or increasing the half life of the agent, thus resulting in a significantly higher amount of a released agent at a target site (e.g., a tumor or liver (e.g., cirrhotic cell). According, the conjugates and particles disclosed herein can be more effective therapeutically than the free agent (e.g., due to enhanced drug uptake in the target tissue) and/or allow for a lower therapeutic dose of the agent, e.g., without substantially compromising the resulting drug concentration at a target tissue. In some embodiments, the conjugates and particles disclosed herein can reduce the adverse effect associated with systemic administration of an agent in free form (e.g., not coupled to a polymer, conjugate or particle described herein).

Without being bound by theory, due to the localized delivery of the compositions described herein (e.g., the agent-containing particles), a lower dose or amount of the agent in the particles can be administered (e.g., through local sustained delivery) compared to the agent in free form. In other embodiments, the agent-containing particles are administered at a dose or amount of the agent that is less than the dose or amount of said agent in free form to have a desired effect (e.g., a desired therapeutic effect).

In some embodiments, the agent is incorporated into a particle at a dose that is less than the dose or amount of said agent in free form to have a desired effect (e.g., a desired therapeutic effect), e.g., the standard of care dose for the intended use of the free agent. In one embodiment, the agent are incorporated into the particles at a dose or amount of the agent that is less than the standard of care dose of the agent for a desired therapy (e.g., a dose that is less than about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, or about 0.95 that of the standard of care dose of the agent).

In some embodiments, the agent is incorporated into a particle at a dose equivalent to the dose or amount of said agent in free form to have a desired effect (e.g., a desired therapeutic effect), e.g., the standard of care dose for the intended use of the free agent. In these embodiments, the particle produces a greater therapeutic effect and/or a less adverse effect than the free agent. In certain embodiments, the particle increases the amount of the agent delivered to a tissue or cell in need thereof and reduces the amount of the agent exposed to a non-target tissue or cell, as compared to the free agent.

In some embodiments, the agent is incorporated into a particle at a dose higher than the dose or amount of said agent in free form to have a desired effect (e.g., a desired therapeutic effect), e.g., the standard of care dose for the intended use of the free agent. In some embodiments, the agent is incorporated into a particle at a dose higher than the dose or amount of said agent in free form that would produce an adverse effect by systemic administration (e.g., a reduction in blood pressure). In some embodiments, since the particle described herein releases the agent at a target site based on pH microenvironment, other non-target sites (e.g., blood vessels) with different pH would be less likely to be exposed to the agent.

In another aspect, provided are kits including a first container comprising a polymer or composition described herein and instructions for use. The kits may further comprise a container (e.g., a vial, ampule, bottle, syringe, and/or dispenser package, or other suitable container). In some embodiments, provided kits may optionally further include a second container comprising an excipient for dilution or suspension of a composition or polymer described herein. In some embodiments, the composition described herein provided in the first container and the second container are combined to form one unit dosage form.

In certain embodiments, the kits are useful for delivering an agent (e.g., to a subject or cell). In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for delivering an agent. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods of Treatment and Uses

The present disclosure also provides methods of using the compositions described herein, or a pharmaceutical composition thereof, for delivering an agent. The present disclosure also provides methods of using the polymers described herein, or a pharmaceutical composition thereof, for the treatment, prevention, or diagnosis of a disease or condition.

In certain embodiments, the methods described herein include treating a disease, disorder, or condition from which a subject suffers, comprising administering to a subject in need thereof an effective amount of a composition described herein. In certain embodiments, the methods described herein include implanting in a subject an effective amount of the composition described herein. In certain embodiments, the methods described herein comprise treating a disease or condition in a subject in need thereof by administering to or implanting in the subject a therapeutically effective amount of a composition. In certain embodiments, the methods described herein comprise preventing a disease or condition in a subject in need thereof by administering to or implanting in the subject a prophylactically effective amount of a composition. In certain embodiments, the methods described herein comprise diagnosing a disease or condition in a subject in need thereof by administering to or implanting in the subject a diagnostically effective amount of a composition.

In certain embodiments, the disease or condition is a genetic disease, proliferative disease, hematological disease, neurological disease, painful condition, psychiatric disorder, metabolic disorder, long-term medical condition, cancer (e.g. lung cancer, large bowel cancer, pancreas cancer, biliary tract cancer, or endometrial cancer), neoplasm, angiogenesis, inflammatory disease, autoinflammatory disease, liver disease, lung disease, spleen disease, familial amyloid neuropathy, cardiovascular disease, viral infection, fibrotic condition, or autoimmune disease.

In some embodiments, the compositions are useful in treating lung cancer, head-and-neck cancer, esophagus cancer, stomach cancer, breast cancer, pancreas cancer, liver cancer, kidney cancer, prostate caner, glioblastomas, metastatic melanomas, peritoneal or pleural mesotheliomas.

In some embodiments, the proliferative disease is a benign neoplasm. All types of benign neoplasms disclosed herein or known in the art are contemplated as being within the scope of the disclosure. In some embodiments, the proliferative disease is associated with angiogenesis. All types of angiogenesis disclosed herein or known in the art are contemplated as being within the scope of the disclosure. In certain embodiments, the proliferative disease is an inflammatory disease. All types of inflammatory diseases disclosed herein or known in the art are contemplated as being within the scope of the disclosure. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the disclosure. In some embodiments, the proliferative disease is an autoimmune disease. All types of autoimmune diseases disclosed herein or known in the art are contemplated as being within the scope of the disclosure.

In certain embodiments, the disease is a cardiovascular disease. In certain embodiments, the disease is atherogenesis or atherosclerosis. In certain embodiments, the disease is arterial stent occlusion, heart failure (e.g., congestive heart failure), a coronary arterial disease, myocarditis, pericarditis, a cardiac valvular disease, stenosis, restenosis, in-stent-stenosis, angina pectoris, myocardial infarction, acute coronary syndromes, coronary artery bypass grafting, a cardio-pulmonary bypass procedure, endotoxemia, ischemia-reperfusion injury, cerebrovascular ischemia (stroke), renal reperfusion injury, embolism (e.g., pulmonary, renal, hepatic, gastro-intestinal, or peripheral limb embolism), or myocardial ischemia.

In certain embodiments, the disease is a fibrotic condition. In certain embodiments, the disease is selected from the group consisting of renal fibrosis, post-operative stricture, keloid formation, hepatic cirrhosis, biliary cirrhosis, and cardiac fibrosis. In certain embodiments, the disease is scleroderma. In certain embodiments, the disease is idiopathic pulmonary fibrosis.

In certain embodiments, the methods described herein include contacting a cell with an effective amount of a composition thereof. In certain embodiments, the cell is in vitro. In certain embodiments, the cell is in vivo.

In another aspect, provided herein is a method of delivery an agent to a cell, comprising contacting the cell with a composition as described herein. In certain embodiments, the agent is a polynucleotide. Representative polynucleotides are described herein. In a particular embodiment, the polynucleotide is DNA. In another particular embodiment, the polynucleotide is RNA. In certain embodiments, upon delivery of the RNA into the cell, the RNA is able to interfere with the expression of a specific gene in the cell. In other embodiments, upon delivery of the RNA into the cell, the RNA is able to express a protein. In certain embodiments, the cell is a liver cell, a spleen cell, a lung cell, a heart cell, a kidney cell, or a pancreas cell.

In another aspect, provided herein is a method of delivery an agent to an organ in a subject, comprising administering a composition as described herein to the subject. In certain embodiments, the agent is a polynucleotide. Representative polynucleotides are described herein. In a particular embodiment, the polynucleotide is RNA. In certain embodiments, the organ is the liver, the spleen, the lungs, the heart, the kidneys, or the pancreas.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Experimental Section

### Materials

ε-caprolactone (CL), 5-Dodecanolide (DD), δ-Tetradecalactone (TD), 2-([2-(Dimethylamino)ethyl]methylamino)ethanol (A), N,N,N′,N′-Tetrakis(2-hydroxyethyl)ethylenediamine (B), 2-Dimethylaminoethanol (D), N-Methyldiethanolamine (E) were purchased from Milipore-Sigma (St. Louis, Mo., USA), s-Triazine-1,3,5-triethanol (F) was purchased from Santa Cruz Biotechnology (Dallat, Tex., USA). Custom aminoalcohols, 3,3′,3″-((nitrilotris(ethane-2,1-diyl))tris(methylazanediyl))tris(propan-1-ol) and 3,3′,3″,3′″,3″″,3″″′-((nitrilotris(ethane-2,1-diyl))tris(azanetriyl))hexakis(propan-1-ol) were synthesized according to the procedure below. All the solvents were purchased from Milipore-Sigma at ACS grade. All chemical reagents were used as received with no further purification. In vivo jetPEI was obtained from VWR (Radnor, Pa.). Cy-5 labeled luciferase-encoding mRNA was purchased from TriLink Biotechnologies (San Diego, Calif.).

### General Methods and Instruments

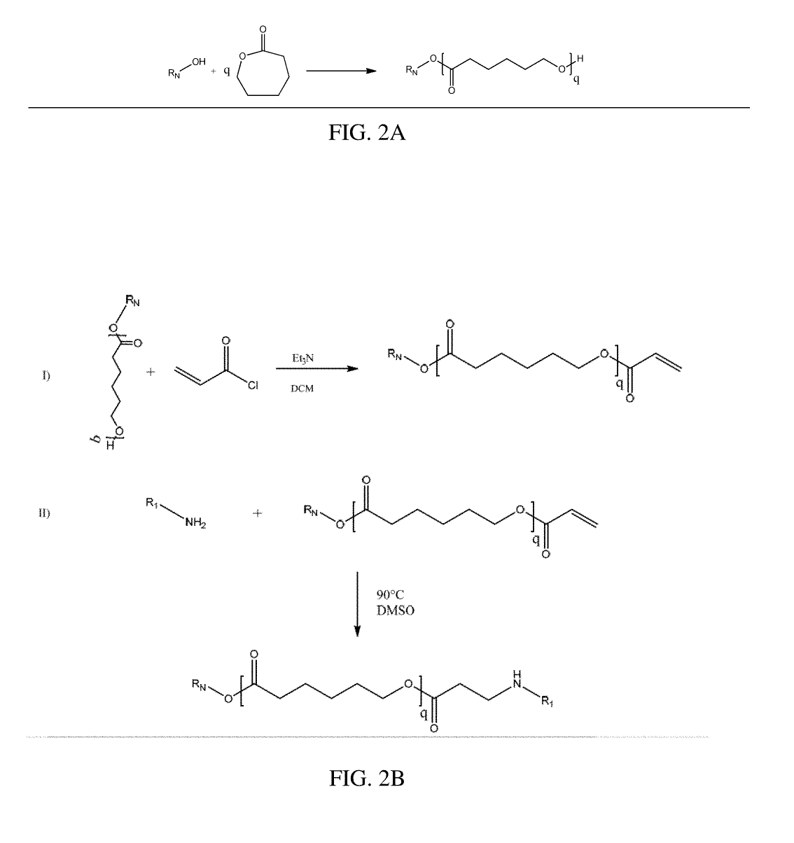

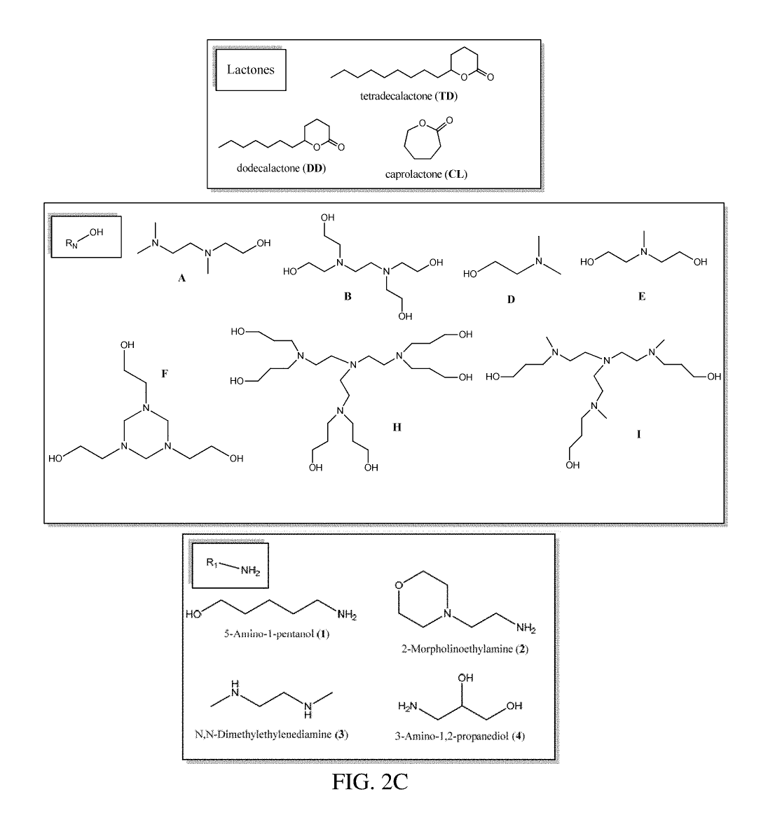

Amino-polyesters were synthesized via Ring Opening Polymerization of selected lactones (5-Dodecanolide (DD), δ-Tetradecalactone (TD), Polycaprolactone (PCL)) initiated by tertiary amino-alcohols (A-1) in the presence of triazabicyclodecene (TBD) catalyst (see FIG. 2A). Polymerization was stopped by the addition of benzoic acid and the polymers were purified by extraction and washing with distilled water and brine. Selected APEs were acylated with acryloyl chloride and subsequently end functionalized via Michael addition with various amines (see FIG. 2B). Amines were fed in large excess in order to avoid affecting the mono-dispersity of the polymers by minimizing the possibility of step-growth polymerization. It is important to note that the list of the amino-alcohols, lactones and amines are not exhaustive (see FIG. 2C).

Polymers were characterized using gel permeation chromatography (GPC) and H1-NMR. Library of APEs was formulated into nanoparticles encapsulating Firefly Luciferase (FLuc) mRNA, composed of APE:DOPE:Chol:C14-PEG2000 (LNP) or APE:C14-PEG2000 (PEG), and screened in Hela cells. In vitro cell viability and Flue luminescence were quantified using Multitox-Flour Multuplex cytotoxicity and Brigh-Glo luciferase assays. Size of the nanoparticles was determined using dynamic light scattering (DLS). The efficacy of mRNA encapsulation into APE nanoparticles was analyzed using Ribogreen reagent or Nanodrop spectrophotometer. For in vivo studies, APE nanoparticles containing FLuc mRNA were injected i.v. into C57BL/6 mice at 0.7 mg/kg. Mice were sacrificed 6 h after nanoparticle administration and the luminescence from organs was detected using IVIS imaging system.

### Example 1: Synthesis of the PCL Based APEs

The PCL based APEs were synthesized via ring opening polymerization of caprolactone (CL) in the presence of different amino alcohols as initiators and TBD as catalyst in THF at 0° C. The monomer to the initiator hydroxyl group ratio was set equal to 3 and 5 in order to obtain APEs with 3 and 5 units of lactones for each arm, respectively. The hydroxyl group of the initiator to catalyst molar ratio was set equal to 10. As an example, for B-CL3, 0.57 g of CL, 0.1 g of B, and 12 mg of Na₂SO₄ were dissolved in 0.57 g of THF and left to stir at 0° C. for 15 min. Then the mixture was poured in a round bottom flask with 24 mg of TBD and 12 mg of Na₂SO₄ and was left to react at 0° C. for 4 h under vigorous stirring. The polymerization was stopped by adding an excess of a solution of benzoic acid in THF (1 mmol/mL). The final mixture was dried under vacuum, dissolved in diethyl ether and washed several times with deionized water and brine. The organic phase was recovered, dried with Na₂SO₄, filtered and dried under vacuum. In the case of B-CL3, aliquots of the mixture were taken after 2.5, 5, 10, 20, 40, 60, 120 and 240 min and poured in an excess of benzoic acid solution in THF. All the aliquots of the B-CL3 as well as all the final APEs before and after the purification were characterized via GPC and ¹H-NMR (CDCl₃, Bruker, 400 Mhz). Polylactone-based diacrylates and PBAE polymers were dissolved in tetrahydrofuran (THF) at a concentration of 4 mg ml⁻¹, filtered over 0.2 um PTFE syringe filter and eluted in Styragel columns at a 1 ml ml⁻¹ flow rate. The instrument is equipped with a Malvern Viscotek™ TDA 305 triple detector. Molecular weights and polydispersities were relative to linear polystyrene standards.

### Example 2: Synthesis of the DD and TD Based APEs

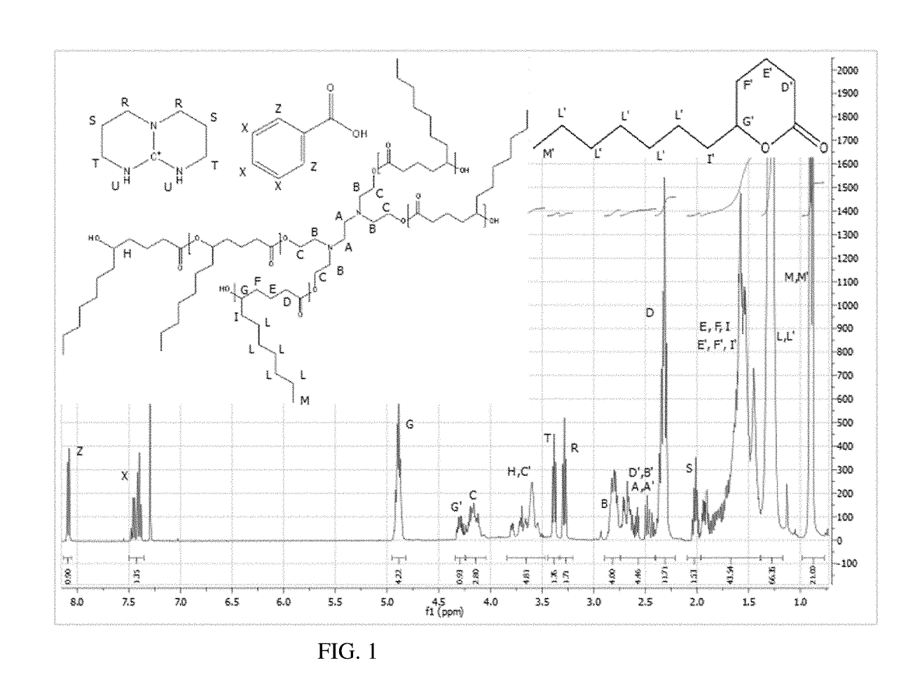

The DD or TD based APEs were synthesized via ring opening polymerization of TD or DD in the presence of different amino alcohols as initiators and TBD as catalyst in bulk at room temperature. The monomer to the initiator hydroxyl group ratio was set equal to 3 and 5 in order to obtain APEs with 3 and 5 units of lactones for each arm, respectively. The hydroxyl group of the initiator to catalyst molar ratio was set equal to 10. As an example, for B-DD3, 2.51 g of DD, 0.25 g of B, and 40 mg of Na₂SO₄ were poured in a 10 ml vial and left to stir for 15 min. Then the mixture was poured in a round bottom flask with 59 mg of TBD and 40 mg of Na₂SO₄ and was left to react under vigorous stirring for 24 h. The polymerization was stopped by adding an excess of benzoic acid in diethyl ether (1 mmol/mL). The final mixture was further diluted in diethyl ether, washed several times with deionized water and brine. The organic phase was recovered, dried with Na₂SO₄, filtered and dried under vacuum. In the case of B-DD3 and B-TD3, aliquots of the mixture were taken after 1, 2, 3, 4, 6, 24 h and poured in an excess of benzoic acid solution in diethyl ether. All the aliquots of the B-DD3/B-TD3 as well as all the final APEs before and after purification were characterized via GPC and ¹H-NMR (CDCl₃, Bruker, 400 Mhz). An example of ¹H-NMR and proton assignment for B-DD3 is reported in FIG. 1.

### Example 3: Instrumentation and Characterization

Molecular weight and polydispersity (Ð) of the polymers were determined by Gel Permeation Chromatography (GPC) carried out in tetrahydrofuran (THF) mobile phase calibrated with linear polystyrene standards on viscotek LT6000L columns, operating at 1.0 mL/min with a Malvern Viscotek™ TDA 305 triple detection system. Samples were filtered through 0.2 μm PTFE filters (WVR) before injections and at approximately 1 mg/mL polymer concentration. ¹H NMR spectra were recorded on a Bruker AVANCE-400 400 MHz NMR spectrometer in deuterated chloroform (CDCl₃, Milipore-Sigma), using the residual proton resonance of the solvent peak at 7.26 ppm as the internal standard. Chemical shifts are reported in parts per million (ppm). Cryo-Transmission Electron Microscopy was performed using JEOL 21 OOF transmission electron microscope operating at 120-200 kV. Specimens were frozen in liquid ethane and mounted in a Gatan cryo-stage. Images were recorded under low-dose conditions with a slow-scan CCD camera.

### Example 4: mRNA Synthesis

Firefly luciferase (FLuc) and Scrambled mRNAs were synthesized by an in vitro transcription from a DNA template as described previously²². Final, purified mRNAs contained a 5′ cap (Cap1), a 5′ UTR consisting of a partial sequence of the cytomegalovirus (CMV) immediate early 1 (IE1) gene, a coding region as described below, a 3′ UTR consisting of a partial sequence of the human growth hormone (hGH) gene, and a 3′ polyA tail estimated to be approximately 100 nucleotides long.

FLuc:
(SEQ ID NO: 1)
AUGGAAGAUGCCAAAAACAUUAAGAAGGGCCCAGCGCCAUUCUACCCACU
CGAAGACGGGACCGCCGGCGAGCAGCUGCACAAAGCCAUGAAGCGCUACG
CCCUGGUGCCCGGCACCAUCGCCUUUAGACGCACAUAUCGAGGUGGACAU
UACCUACGCCGAGUACUUCGAGAUGAGCGUUCGGCUGGCAGAAGCUAUGA
AGCGCUAUGGGCUGAAUACAAACCAUCGGAUCGUGGUGUGCAGCGAGAAU
AGCUCAGUUCUUCAUGCCCGUGUUGGGUGCCCUGUUCAUCGGUGUGGCUG
UGGCCCCAGCUAACGACAUCUACAACGAGCGCGAGCUGCUGAACAGCAUG
GGCAUCAGCCAGCCCACCGUCGUAUUCGUAGAAGAAAGGGCUGCAAAAGA
UCCUCAACGUGCAAAAGAAGCUACCGAUCAUACAAAAGAUCAUCAUCAUG
GAUAGCAAGACCGACUACCAGGGCUUCCAAAGCAUGUACACCUUCGUGAC
UUCCCUUUCCACCCGGCUUCAACGAGUACGACUUCGUGCCCGAGAGCUUC
GACCGGGACAAAACCAUCGCCCUGAUCAUGAACAGUAGUGGCAGUACCGG
AUUGCCCAAGGGCGUAGCCCUACCGCACCGCCCGCUGUGUCCGAUUCAGU
CAUGCCCGCGACCCCAUCUUCGGCAACCAGAUCAUCCCCGACACCGCUAU
CCUCAGCGUGGUGCCAUUUCACCACGGCUUCGGCAUGUUCACCACGCUGG
GCUACUUAUCUGGGCUUUCGGGUCGUGCUCAUGUACCGCUUCGAGGAGGA
GCUAUUCUUGCGCAGCUUGCAAGACUAUAAGAUUCAAUCUGCCCUGCUGG
UGCCCACACUAUUUAGCUUCUUCGCUAAGAGCAUCUCAUGACAAGUACGA
CCUAAGCAACUUGCACGAGAUCGCCAGCGGCGGGGCGCCGCUCAGCAAGG
AGGUAGGUGAGGCCGUGGCCAAACGCUUCCACCUACCAGGCAUCCGCCAG
GGCUACGGCUGACAGAACAACCAGCGCCAUUCUGAUCACCCCCGAAGGGG
ACGACAAGCCUGGCGCAGUAGGCAAGGUGGUGCCCUUCUUCGAGGCUAAG
GUGGUGGACUUGGACACCGGUAAGACACUGGGUGUAACCAGCGGGCGAGC
UGUGCGUCCGUGGCCCCAUGAUCAUGAGCGGCUACGUUAACAACCCCGAG
GCUACAAACGCUCUCAUCGACAAGGACGGCUGGCUGCACAGCGGCGACAU
CGCCUACUGGGCGAGGACGAGCACUUCUUCAUCGUGGACCGGCUGAAGAG
CCUGAUCAAAUACAAGGGCUACCAGGUAGCCCCAGCCGAACUGGAGAGCA
UCCUGCUGCAACACCCCAACAUCUUCGACGCCGGGGUCGGGCCUGCCCGA
CGACGAUGCCGGCGAGCUGCCCGCCGCAGUCGUCGUGCUGGAACACGGUA
AAACCAUGACCGAGAAGGAGAUCGUGGACUAUGUGGCCAGCCAGGUUACA
ACCGCCAAGAAGCUGGGGUGGUGUUGUGUUCGUGGACGAGGUGCCUAAAG
GACUGACCGGCAAGUUGGACGCCCGCAAGAUCCGCGAGAUUCUCAUUAAG
GCCAAGAAGGGCGGCAAGAUCGCCGUGUAA
Scrambled:
(SEQ ID NO: 2)
TAACGGCCACCTACCTTATAGATCGGCAGCCTTAACAATTCCCAACCTTC
TCGTGCTCGTGATTGGCCCCCCATGAGCATTCAATAAGAGGGTACACGGG
TCTTGGTCAAGGTCAGAGAGAAACAGAAATGCGATCCCGCTGAGTCACCG
ATATCCGTTGGCCAGCCAGTGCCCGTTGGGAGTCCGCACAGCAAAGAGCG
CTTCGTCACACGAAACGGGCAAATTGTATCAGATGAACGCCTTCCGCAAA
GACGACGCGCCACAAGGAAGCTGAGCTCTAAGGTTACCAATAACGGCTGT
AGGCCCCTAGCTTTCTCCTGCGCCCAAATAACCGCTAACGCGGCTATGCC
TCGCACGGTTTTAACTTGGTACGGGCATCTCAGATTTGGCGCCCCGGATT
AGCATCTCGGGCGCTTCAGCCCGGCATCTATAAAGGAGAGCCACACCGAG
ATCCCGTGGAgtGCCAGATCGTTTCGCATAAGGGGGGGCCGACTGGGGGA
AGCGCCAAGACGGGGCGGCGGCCGCAGCCAATGTACCCGGCTCAAATGCC
TTATCCGTGCCCTGACACGACCAACAAGTGCCGAGAAGCTATGCCGGGTA
TAGCGACACCCGCGAACGATTACGGTGCGACTAGCATTATTACCTGCTCA
TGGGGCAACTCAACTTGAACAACTCATCCCTCCGCCCCGCAGCCCGCTAG
ATACTGGAGATATTCAGCACAGGAAGCGCTCCTGAGCTTTCACTCCCTGC
TGGGGGGGCCAATATCCATAAAGGACCTGAGTGGAGATATCCGGGCAGCC
GGACTGGGGGACGTGACGACAACTATACGCAGATTTAGCCGCCGAGGGCC
CGCGAAAGCCGGGTCACCGCCCCCTTCTATCTCCCTAG

### Example 5: Nanoparticle Formulation

The APE lipid nanoparticles (APE-LNPs) were prepared by mixing ethanol and aqueous phase at a 1:3 ratio in a microfluidic chip device using syringe pumps or in a 96-well plate with a magnetic steering for a high-throughput APE-LNP library screen. The ethanol phase was prepared by solubilizing a mixture of ionizable amino-polyester, 1,2-dioleoyl-sn-glycero-3-phosphoethanolamine (DOPE, Avanti), cholesterol (Sigma), and 1,2-dimyristoyl-sn-glycero-3-phosphoethanolamine-N-[methoxy-(polyethyleneglycol)-2000] (ammonium salt) (C14-PEG 2000, Avanti) at a molar ratio of 50:25:23.5:1.5 and a 8:1 Nitrogen to Phosphate (N/P) ratio. The efficacy of mRNA encapsulation into APE nanoparticles was analyzed using Ribogreen reagent or Nanodrop spectrophotometer (Thermo-Fisher Scientific). NP Size, PDI, and C, potential were determined using dynamic light scattering (DLS) with a Zetasizer (Malvern Instruments). All the values reported are an average of three independent measurements and are relative to the volume.

### Example 6: In Vitro Transfections

HeLa cells (ATCC® CCL-2™) were cultured in Dulbecco's Modified Eagle's Medium (4500 mg/L glucose) supplemented with 10% heat-inactivated fetal bovine serum (hiFBS, Gibco) and penicillin/streptomycin at 37° C. and 5% CO₂. Cells were passaged every 3-4 days. For the APE library screen, 10,000 cells/200 μL per well were seeded in a 96-well plate (Costar), one day before the experiment. APE-LNPs containing 100 ng of Flue mRNA were added to each well and incubated for 24 h. Cell Viability and Flue luminescence were analyzed on Tecan Infinite M200 plate reader (Tecan US, Morrisville, N.C.) using Multitox-Flour Multiplex cytotoxicity and Bright-Glo luciferase assays (Promega) according to manufactures protocol.

### Example 7: Cellular Uptake Analysis

For the flow cytometry analysis, Hela cells were seeded at 90,000 cells/500 μL per well into 24-well plates one day before the experiment and incubated for 24 h with the APEs containing 250 ng of Cy5 labeled mRNA. Cell cells were washed with PBS and detached from the surface using trypsin/EDTA (Sigma, Ayrshire, UK) after which they were immediately transferred to tubes containing 5% FBS (fetal bovine serum, Thermo Scientific) in PBS and kept on ice. Next, samples were centrifuged for 5 min at 220 g at 4° C., followed by two washing steps with 3 mL of 5% FBS in PBS and suspended in 0.3 mL PBS with SytoxBlue (ThermoFisher) dead cell stain. Samples were analyzed using LSR IIHTS-1 (BD Bioscience) flow cytometer.

For fluorescent microscopy analysis, Hela cells were seeded onto cover glasses placed into 24-well plates at 25,000 cells/500 μL per well one day before the experiment. Cells were incubated for 24 h with the APEs containing 250 ng of Cy5 labeled mRNA. Subsequently, cells were washed with PBS and fixed 4% PFA for 20 min. Cover glasses were washed with PBS, removed from the wells and mounted on the glass slides using Aqua-Poly-Mount (Polysciences) containing DAPI nuclei stain (ThermoFisher). Slides were stored at 4° C. and imaged using LSM 700 Laser Scanning Confocal microscope (Zeiss).

### Example 8: In Vivo Experiments

All animal studies were approved by the MIT Institutional Animal Care and Use Committee and were consistent with local, state, and federal regulations as applicable. For in vivo studies, APE nanoparticles containing FLuc mRNA were injected i.v. into C57BL/6 mice at 0.6 mg/kg. Mice were sacrificed 6 h after nanoparticle administration and the luminescence from organs was detected using IVIS imaging system.

### Example 9: Improved Acryloylation of the Amino-Polyester

An acrylate group was added to the hydroxyl group present at the end of the polymer structure. ATD3 was dissolved in chloroform. The polymer solution was cooled to 0° C. in an ice bath under stirring and then 1.8 equivalent of triethylamine (TEA) was injected through the stopper. Then 1.6 equivalent of acryloyl chloride was fed dropwise with a syringe. As an example, for the acrylation of 5.65 g of polymer, 1.71 ml of TEA and 0.88 ml of acryloyl chloride were added consequently in this order. After the complete feeding of the acryloyl chloride, the reaction is left to equilibrate (and to react) at room temperature (just remove the flask from the ice bath after the injection of acryloyl chloride and leave it under stirring for 1 h).

A purification was performed to eliminate all the TEA salts formed as by-product of the functionalization reaction. Firstly, the polymer solution was filtered using a paper filter to remove most of the TEA salts, then the filtrate polymer solution was poured into a separation funnel in presence of with 0.1 M HCl and brine up to reach 50/50 v/v organic/water solution to allow the phase separation. The up-laying liquid, consisting in water and salts, was disposed, while the down-laying one, consisting in an organic solution of acrylated polymer in chloroform, was recovered. The organic phase was washed with a carbonate buffer and brine. The organic mixture was put in a beaker with some sodium sulfate and a magnetic stirrer; it was kept at room temperature under constant stirring for few minutes to be sure to eliminate potential water drops present in the organic solution after the phase separation. The organic mixture was filtered again using a paper filter, then dried under vacuum to allow the complete evaporation of the organic solvent (chloroform).

### Example 10: Synthesis of the End-Functionalized Amino-Polyester

The acrylated amino-polyester was functionalized via Michael addition of a hydrophilic amine (A1, A2, A3, A4, A5, A6 or A7) or an alkyl amine (C12, C14, C16 or C18) to the vinyl residue at the end of the arm of the acrylated polyester. Each amine, (with an amine to acrylated polymer molar ratio equal to 2) was mixed with 1 ml of methanol/chloroform 50/50 v/v solution in a vial equipped with a stir bar. The amine to acrylated polymer ratio was set equal to 2 to guarantee an excess of amine during the reaction. The choice to use an excess of amines was done in order to avoid the production of dimers and trimers of APEs as in a Michael step-growth polymerization.

Briefly, 100 mg of dried polymer were weighted in a vial. Finally, the amine solution was poured into the vial containing the acrylated polymer and the reactions was carried out for 24 hours at room temperature under constant stirring. As an example, for ATD3-A1, 21.36 mg of N,N′-Dimethylethylenediamine (A1) were dissolved in 1 ml of methanol/chloroform 50/50 v/v solution, then added to 100 mg of ATD3-Ac and left to react at room temperature for 24 hours, under constant stirring. After 24 h evaporate the solvent using Genevac (low boiling point) or using flowing N2 stream and further dry under vacuum. Analyze by GPC (only those soluble in THF) and NMR (chloroform).

### DISCUSSION

A library of 33 APEs (FIGS. 2A-2C) was synthesized via ROP of three lactones (TD, DD, CL) in the presence of several amino-alcohols (A, B, D, E, G, H, I) as initiator and TBD as catalyst. The lactone to initiator molar ratio that represents the desired degree of polymerization (q) was set equal to 3 and 5 in order to study the effect of the polyester arm length and lipophilicity on the transfection efficacy. In the same way, a lactone with no side chain (CL) and two with side chains of different dimensions (TD, DD) have been chosen. To further improve the libraries, several initiators with different number of ionizable amines and different alkoxy groups have used as long as it is well proven in literature that the charge density and the architecture (i.e., linear vs branched) of the carriers are important factors that influence the efficacy of gene delivery carriers.

As visible in FIG. 3A, the ROP of the three different lactones in the presence of an initiator that bears four alkoxy groups and two tertiary amines presents the classic behavior of a controlled polymerization as long as the average-number molecular weight (Mn) is a linear function of the conversion.

In addition, all the synthesized APEs present narrow molecular weight distributions (Ð<1.4) and an Mn close to the theoretical one, as visible in Table 1 In addition, high conversion of the ROP reaction was reached (e.g. X_{CL}>80% for B-CL3) and q is very similar to the designed values (e.g. q_{NMR}=2.78 for B-CL3), as visible in ¹H-NMR reported in FIG. 3B.

Amino-polyesters can be formulated with mRNA and additional helper lipids (e.g. DOPE, Choi, PEG-lipid) into lipid nanoparticles (LNP). A library of LNP-formulated APEs was screened in HeLa cells to identify most potent candidates for mRNA delivery (FIG. 3C). We found several APEs that were able to effectively deliver mRNA to Hela cells including I-DD3, I-TD3, A-TD3, B-DD3, A-DD5, B-TD3, B-DD5 and H-TD3. We observed that amino-polyesters containing lactone-repeating units with the alkyl side chains (TD, DD) were more effective in transfecting HeLa cells as compared to APEs made from poly-caprolactone (CL). Moreover, increasing number of tertiary-amines of the amino-alcohol had a positive effect on APE transfection efficiency. By contrast, increasing number of lactone repeating units from 3-5 did not improve transfection. The absence of CL based APEs among the top performing candidates may indicate that they are not enough lipophilic to correctly condense with mRNA into NPs. However, the lipophilicity is not the unique parameter that affect the transfection efficacy of these carriers as long as the number of lactones seems to not play a relevant role. It may be possible that in some cases the higher number of lactones may reduce the overall charge density of the NP and, in turn, reduce the mRNA loading and/or their ability to act as proton sponge. This latter phenomenon is in agreement with the fact that the NPs composed of APEs with the higher number of tertiary amines (i.e I, B and A) presents the highest transfection efficacy. In order to better study the effect of the structure, the top performing APE (I-DD3) and two other APEs with different lactones and initiators (A-TD3, B-DD3) were chosen as candidate for further characterizations and in vitro and in vivo studies. As all the APEs, I-DD3, A-TD3, and B-DD3 present monomodal molecular weight distributions, as visible in the raw GPC traces in FIG. 3D, and a high reproducibility. This particular characteristic is of vital importance for the possibility to translate this technology into the clinics and at the end into the market, as long as an almost mono-disperse species is produced and, thus, avoiding complex purification steps and tests to identify the portion of the material that is responsible for the transfection efficacy.

In addition, the top performing APEs presents not only a very good reproducibility and low D, but also the L-NPs obtained from them are mono-disperse with low standards deviations as visible in Table 1. Interestingly, the overall surface charge is different even if the same APE to mRNA ratio has been adopted confirming that the different number of amines and Mn affect the density charge of the carriers.

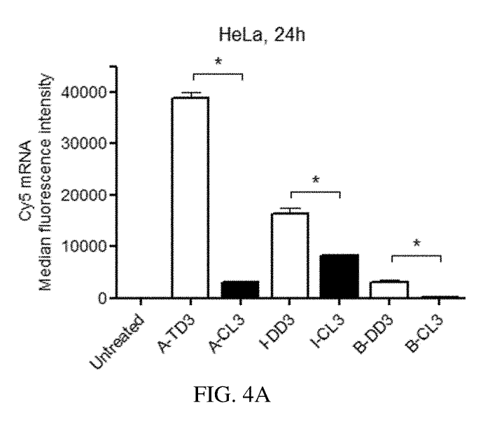

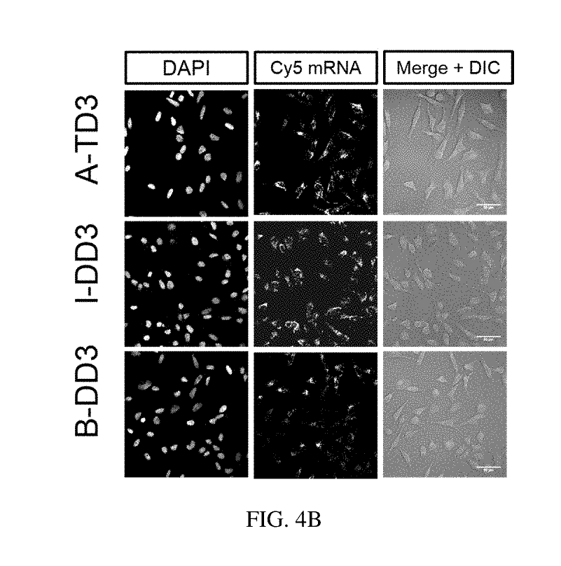

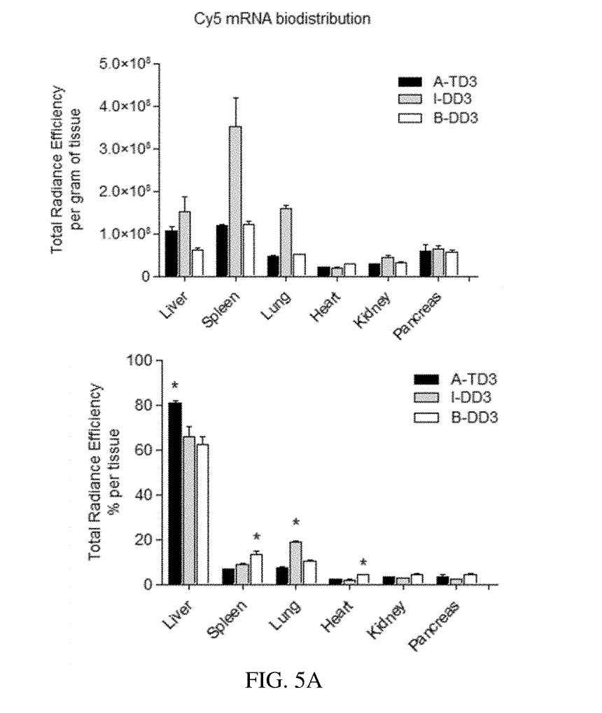

In order to better clarify the motif behind the inability of the PCL-based carriers to transfect, an uptake study has been carried out with the top performing APEs and their corresponding PCL-based ones. As shown in FIGS. 4A-B, all the PCL-based LNPs presents a lower uptake compared to their analogues indicating, that their inability to efficiently cross the cell membrane is the main feature hampering the efficacy of CL-based APEs. This is probably caused by their lower stability in the cell medium that leads to CL-APE NPs aggregation or by the enhance ability of the side alkyl chains of the TD and DD based L-NPs to interact with the cell membranes and facilitate greater uptake. In particular, we found that A-CL3 and B-CL3 nanoparticles were not stable wen stored at 4° C. in PBS and crash out in time.

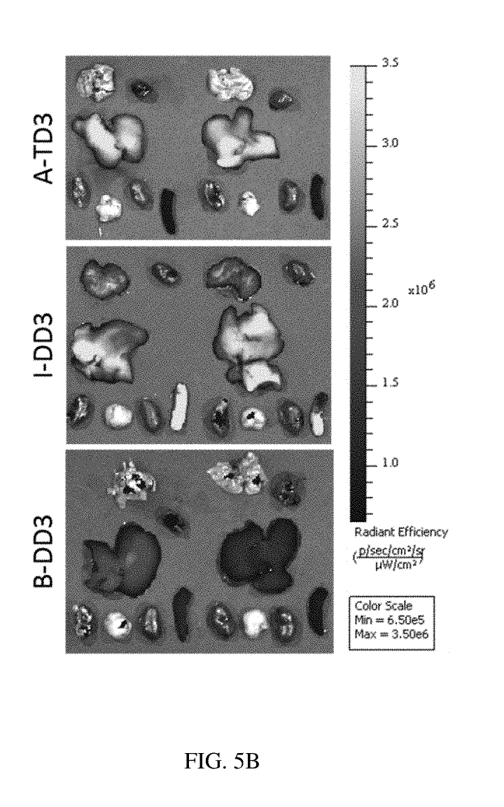

The ability to deliver mRNA of the top performing APEs was further investigated in vivo. APEs were formulated with Cy5 labeled mRNA and injected via tail vein in C57BI/6 mice at 0.6 mg/kg to study the biodistribution of the different NPs in different organs, such lungs, liver and spleen, as visible in FIGS. 5A-B. Interestingly, the distribution of the LNPs vary significantly depending on the polymer composition. A-TD3 based LNPs show preferential accumulation in the liver, while I-DD3 preferentially accumulates in the lungs. B-DD3 shows the highest mRNA accumulation in the spleen.

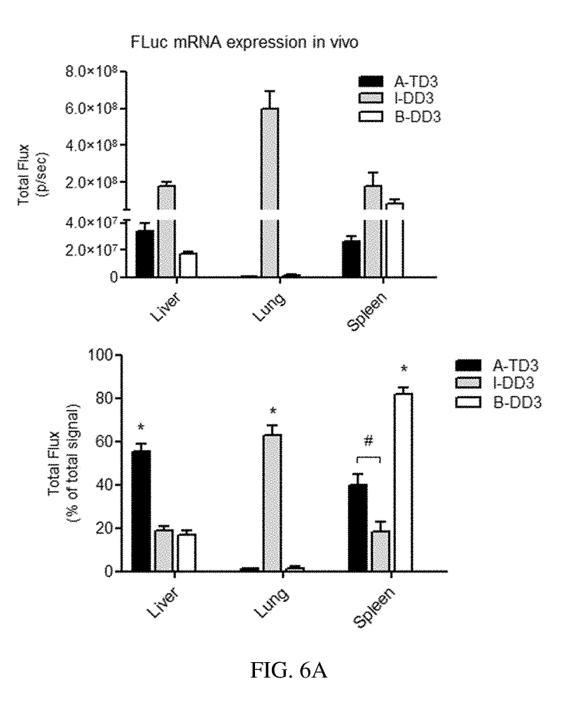

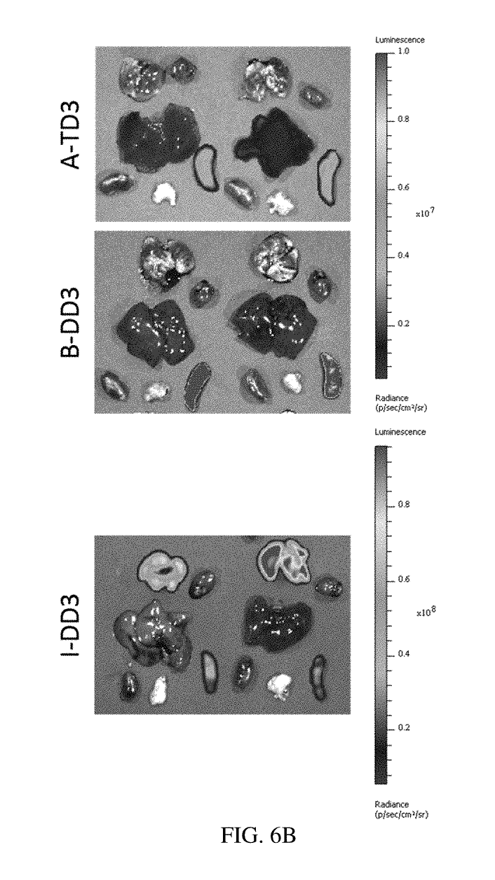

The same pattern seen in the biodistribution is also found when the transfection efficacy of these APEs is studied, as visible in FIGS. 6A-B, by complexing them with Luc-mRNA. A-TD3 based LNPs show preferential transfection in the liver, while I-DD3 preferentially transfect in the lungs and presents the highest mRNA delivery efficacy. On the contrary, B-DD3 preferentially transfect the spleen.

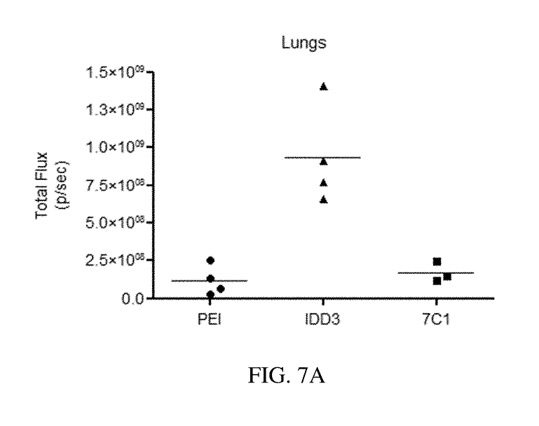

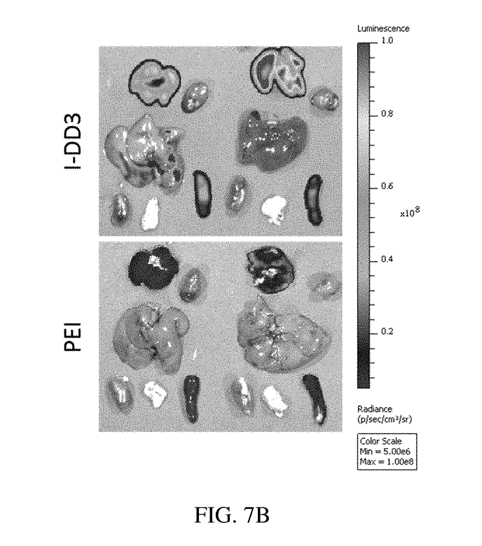

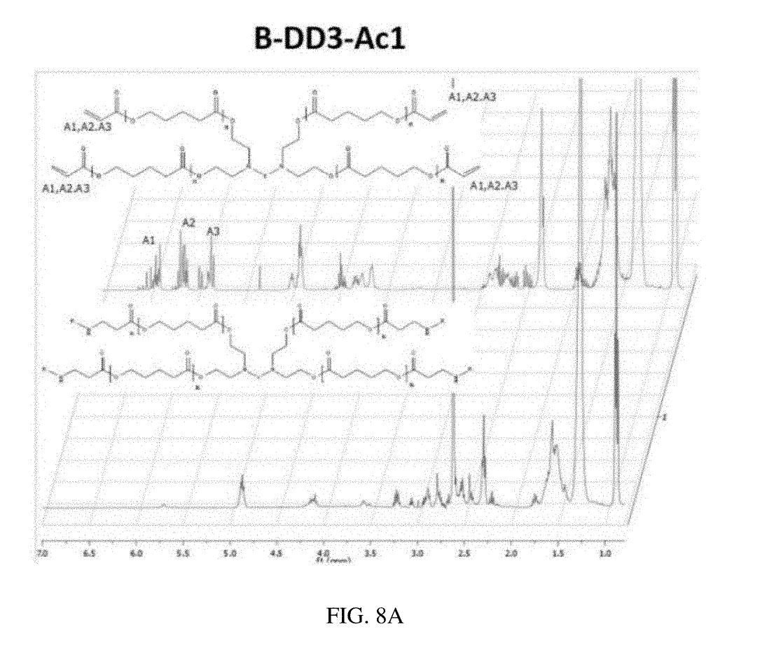

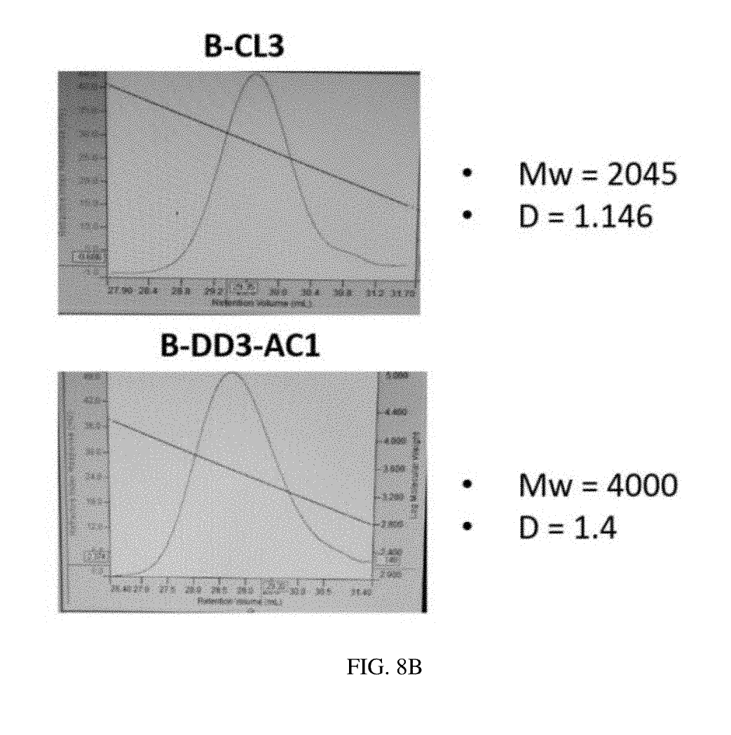

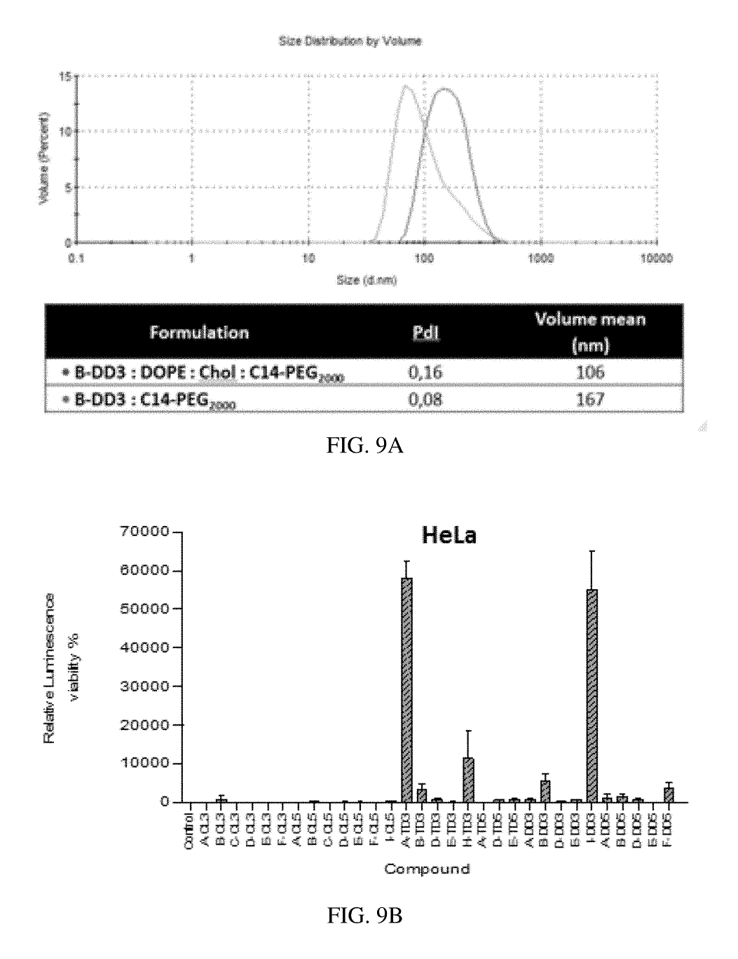

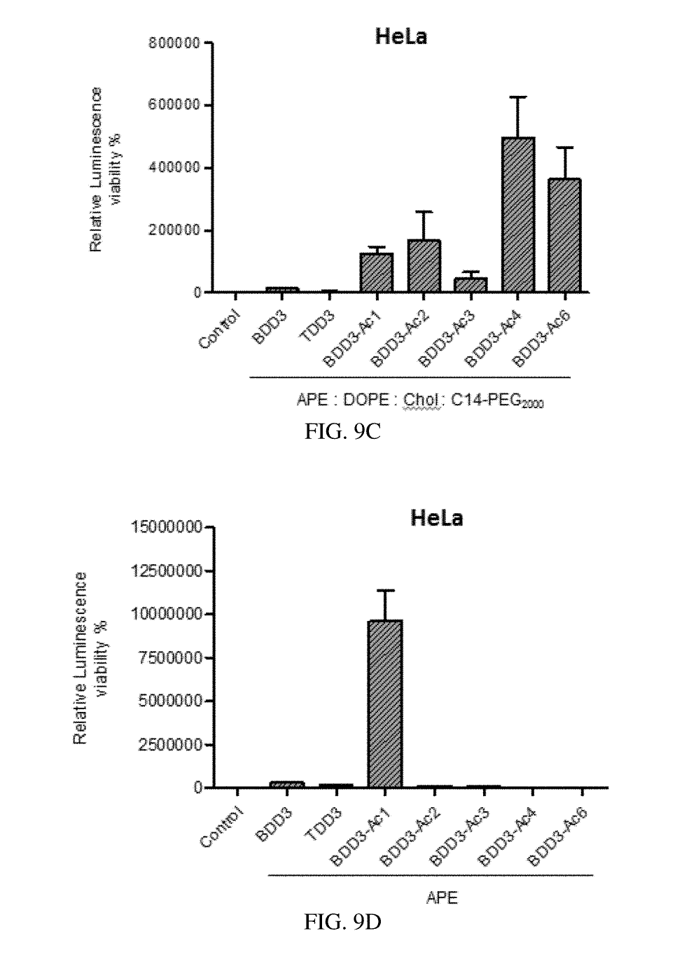

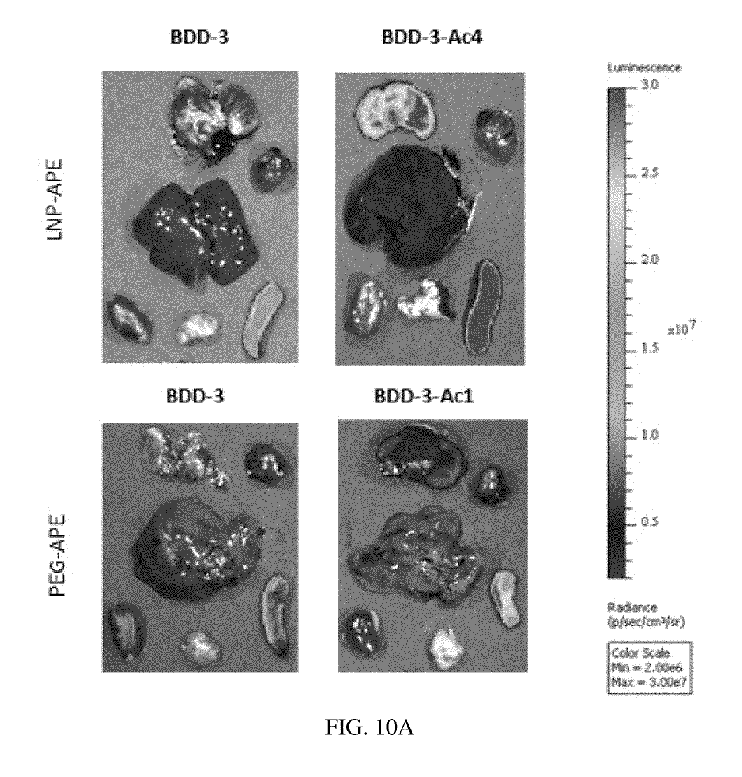

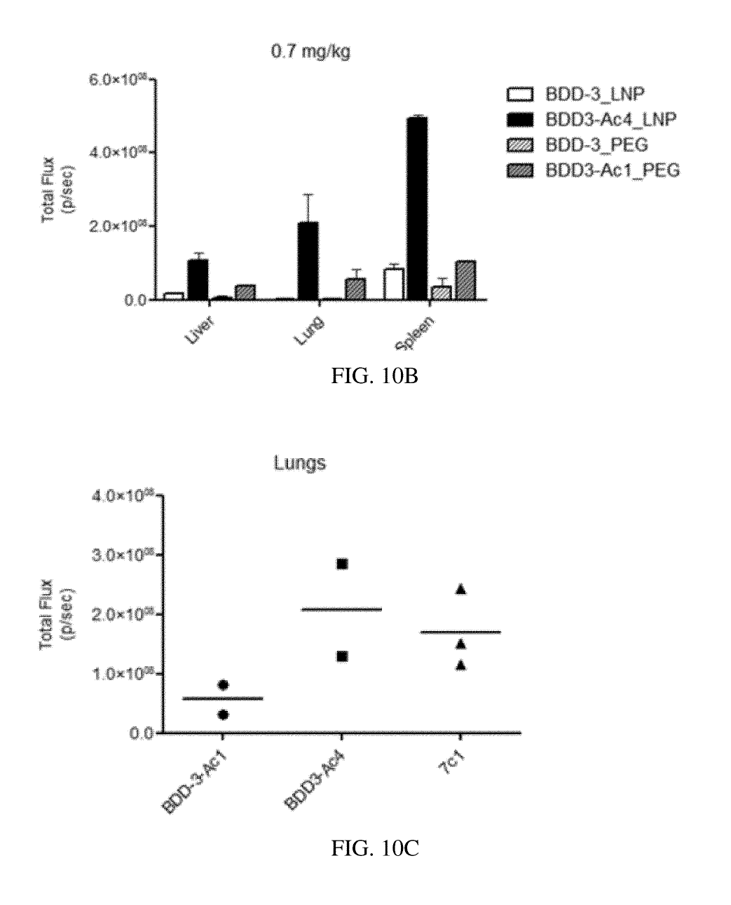

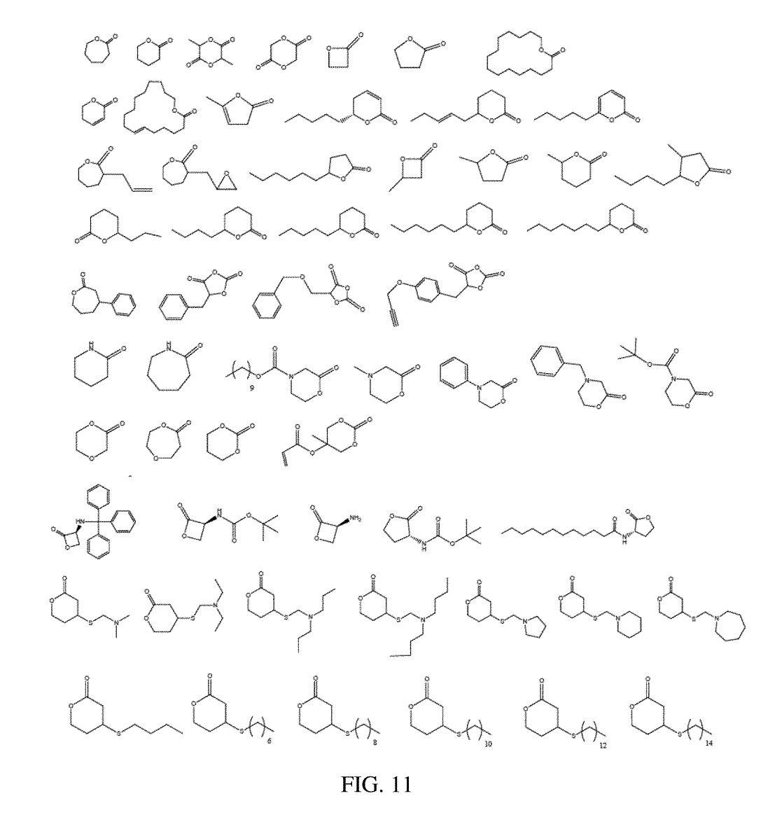

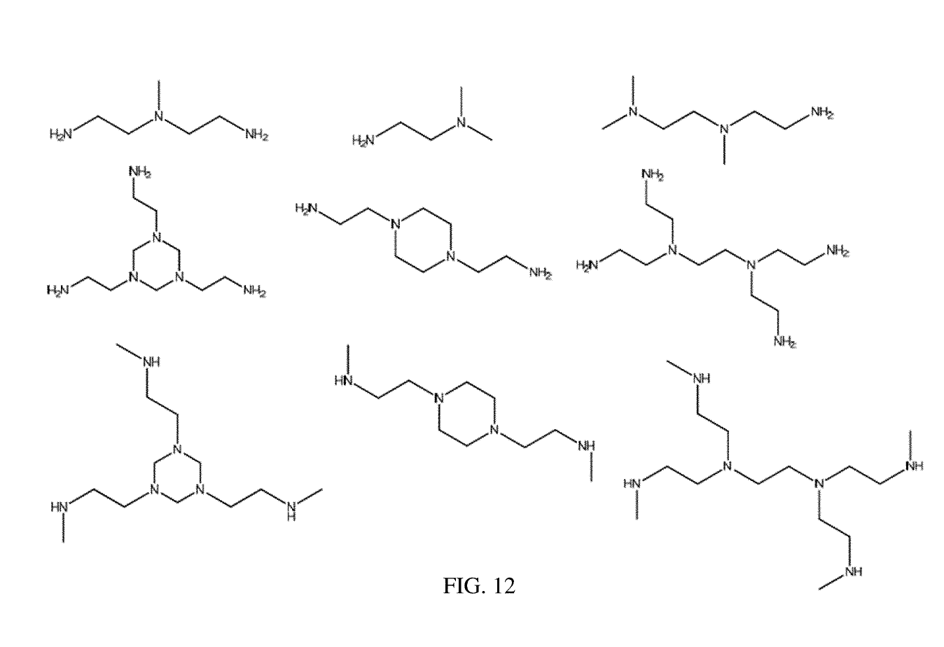

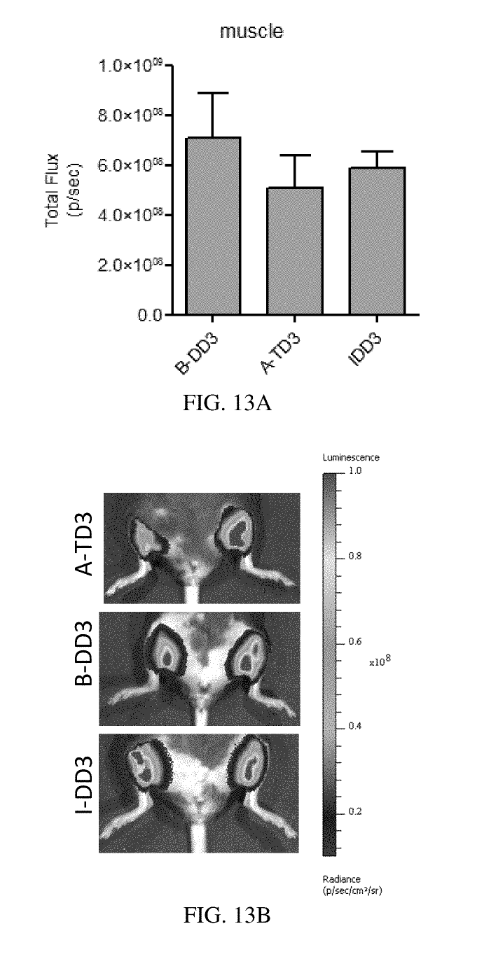

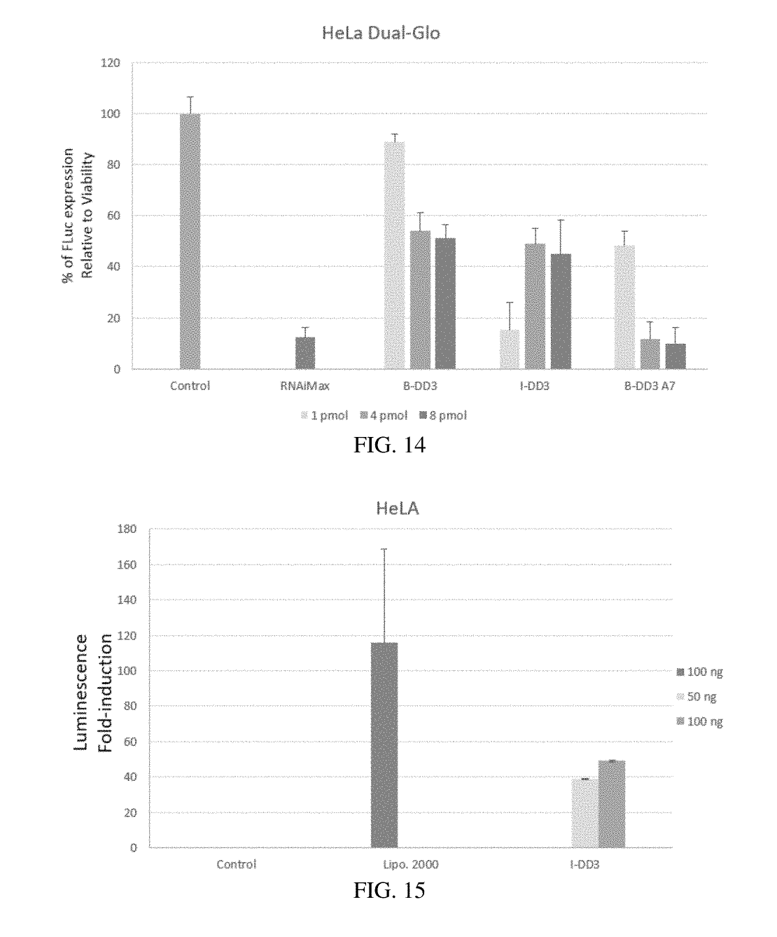

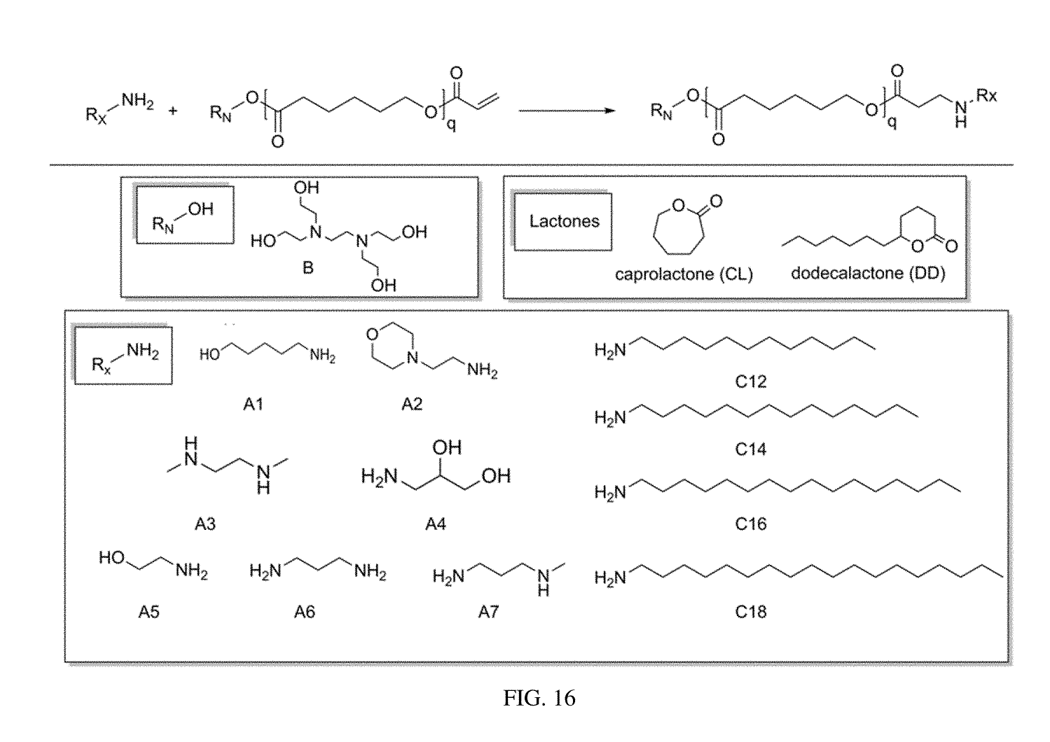

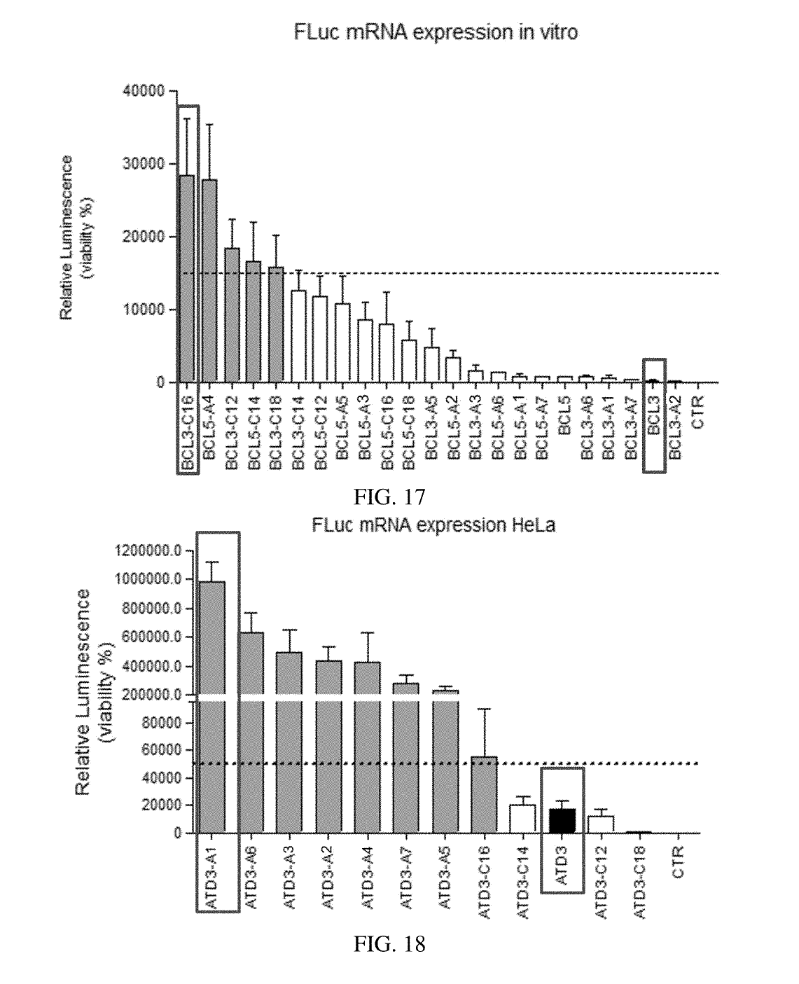

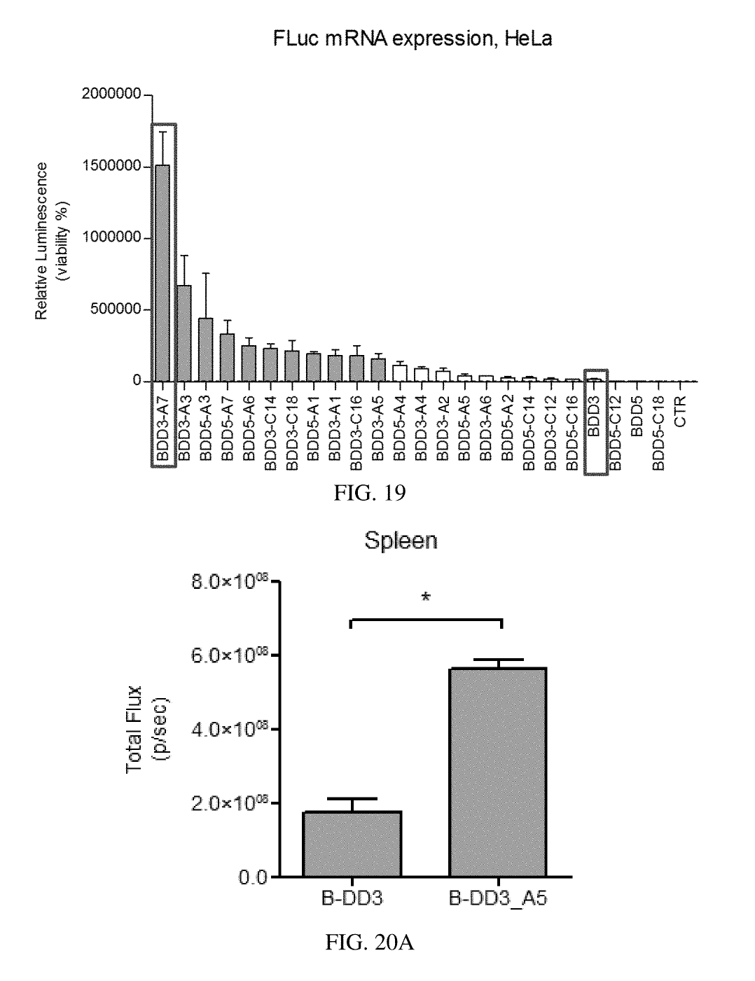

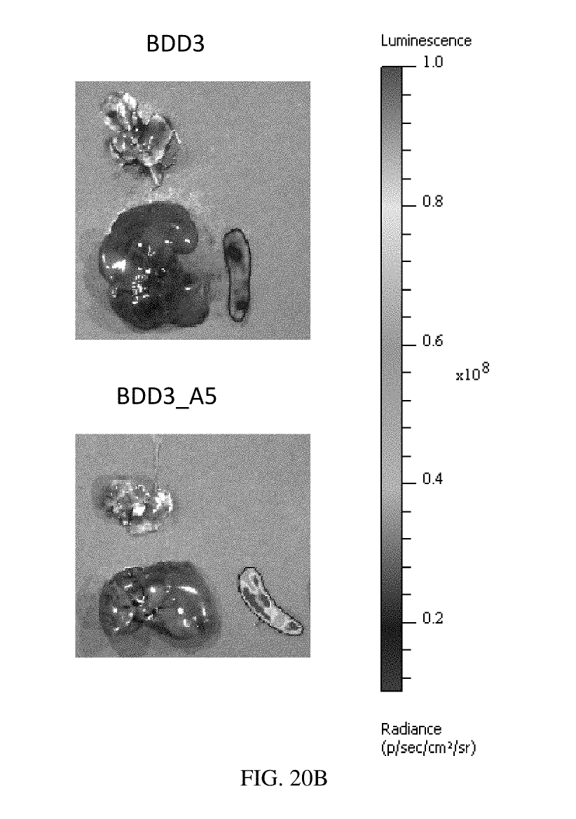

In particular, I-DD3 presents a transfection efficacy one order magnitude higher than PEI in the lungs, as visible in FIGS. 7A-B.

In conclusion, a novel class of gene delivery carriers has been also developed via the ring opening polymerization of different lactones in the presence of several tertiary amino-alcohols. A library of 33 amino-polyesters has been synthesized, formulated with mRNA to form NPs and screened for transfection efficacy in vitro. The top performing NPs have been injected i.v. in mice showing preferential uptake and mRNA transfection in different organs, such as A-TD3 in the liver, I-DD-3 in the lungs, and B-DD3 in the spleen.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### REFERENCES

1. Kamaly, N.; Yameen, B.; Wu, J.; Farokhzad, O. C., Degradable Controlled-Release Polymers and Polymeric Nanoparticles: Mechanisms of Controlling Drug Release. Chemical reviews 2016, 116 (4), 2602-63.
2. Dowdy, S. F., Overcoming cellular barriers for RNA therapeutics. Nature biotechnology 2017, 35 (3), 222-229.
3. Yin, H.; Kanasty, R. L.; Eltoukhy, A. A.; Vegas, A. J.; Dorkin, R. J.; Anderson, D. G., Non-viral vectors for gene-based therapy. Nature Reviews Genetics 2014, 15 (8), 541-555.
4. Davis, M. E., The first targeted delivery of siRNA in humans via a self-assembling, cyclodextrin polymer-based nanoparticle: from concept to clinic. Molecular pharmaceutics 2009, 6 (3), 659-68.
5. Dahlman, J. E.; Barnes, C.; Khan, O. F.; Thiriot, A.; Jhunjunwala, S.; Shaw, T. E.; Xing, Y.; Sager, H. B.; Sahay, G.; Speciner, L.; Bader, A.; Bogorad, R. L.; Yin, H.; Racie, T.; Dong, Y.; Jiang, S.; Seedorf, D.; Dave, A.; Singh Sandhu, K.; Webber, M. J.; Novobrantseva, T.; Ruda, V. M.; Lytton-Jean, A. K.; Levins, C. G.; Kalish, B.; Mudge, D. K.; Perez, M.; Abezgauz, L.; Dutta, P.; Smith, L.; Charisse, K.; Kieran, M. W.; Fitzgerald, K.; Nahrendorf, M.; Danino, D.; Tuder, R. M.; von Andrian, U. H.; Akinc, A.; Panigrahy, D.; Schroeder, A.; Koteliansky, V.; Langer, R.; Anderson, D. G., In vivo endothelial siRNA delivery using polymeric nanoparticles with low molecular weight. Nature nanotechnology 2014, 9 (8), 648-55.
6. Kaczmarek, J. C.; Patel, A. K.; Kauffman, K. J.; Fenton, O. S.; Webber, M. J.; Heartlein, M. W.; DeRosa, F.; Anderson, D. G., Polymer-Lipid Nanoparticles for Systemic Delivery of mRNA to the Lungs. Angew Chem Int Ed Engl 2016, 55 (44), 13808-13812.
7. Lv, H.; Zhang, S.; Wang, B.; Cui, S.; Yan, J., Toxicity of cationic lipids and cationic polymers in gene delivery. Journal of controlled release: official journal of the Controlled Release Society 2006, 114 (1), 100-9.
8. Akhtar, S.; Benter, I., Toxicogenomics of non-viral drug delivery systems for RNAi: potential impact on siRNA-mediated gene silencing activity and specificity. Advanced drug delivery reviews 2007, 59 (2-3), 164-82.
9. Huayu Tian, Z. T., Xiuli Zhuang, Xuesi Chen, Xiabin Jing, Biodegradable synthetic polymers: Preparation, functionalization and biomedical application. Prog Polym Sci 37 (2), 237-280.
10. Jerome, C.; Lecomte, P., Recent advances in the synthesis of aliphatic polyesters by ring-opening polymerization. Advanced drug delivery reviews 2008, 60 (9), 1056-76.
11. Jing Haa, S. E., Daniel J. Siegwart, Progress towards the Synthesis of Amino Polyesters via Ring-Opening Polymerization (ROP) of Functional Lactones. Synlett 2016, 27 (16), 2285-2292.
12. Hao, J.; Kos, P.; Zhou, K.; Miller, J. B.; Xue, L.; Yan, Y.; Xiong, H.; Elkassih, S.; Siegwart, D. J., Rapid Synthesis of a Lipocationic Polyester Library via Ring-Opening Polymerization of Functional Valerolactones for Efficacious siRNA Delivery. Journal of the American Chemical Society 2015, 137 (29), 9206-9.
13. Blake, T. R.; Waymouth, R. M., Organocatalytic ring-opening polymerization of morpholinones: new strategies to functionalized polyesters. Journal of the American Chemical Society 2014, 136 (26), 9252-5.
14. Zhou, J.; Liu, J.; Cheng, C. J.; Patel, T. R.; Weller, C. E.; Piepmeier, J. M.; Jiang, Z.; Saltzman, W. M., Biodegradable poly(amine-co-ester) terpolymers for targeted gene delivery. Nature materials 2011, 11 (1), 82-90.

1. A compound of Formula (I): 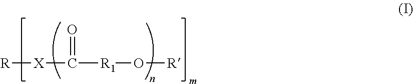
wherein:
X is O, S, or NR₄;
R is optionally substituted heteroaliphatic, optionally substituted heterocyclyl, or a combination thereof, wherein R comprises one or more amine moieties, and wherein all of said amine moieties are tertiary;
each R₁ independently is optionally substituted aliphatic or optionally substituted heteroaliphatic;
R′ is hydrogen or a group of the formula: 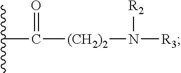
wherein:
each R₂ and R₃ independently is hydrogen, optionally substituted aliphatic, optionally substituted heteroaliphatic, optionally substituted carbocyclyl, optionally substituted heterocyclyl, optionally substituted aryl, or optionally substituted heteroaryl; or
R₂ and R₃ are combined to form an optionally substituted heterocyclyl;
each R₄ independently is hydrogen, optionally substituted aliphatic, or optionally substituted heteroaliphatic;
each n independently is an integer between 1 and 20, inclusive; and
m is an integer between 1 and 10, inclusive.

2. The compound of claim 1, having the structure of Formula (II):

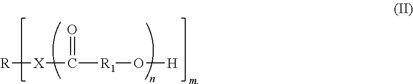

3. The compound of claim 1, having the structure of Formula (III):

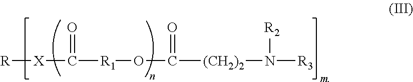

4. The compound of claim 1, wherein R has 1-10 amine moieties.

5. (canceled)

6. The compound of claim 4, wherein R has a structure selected from:

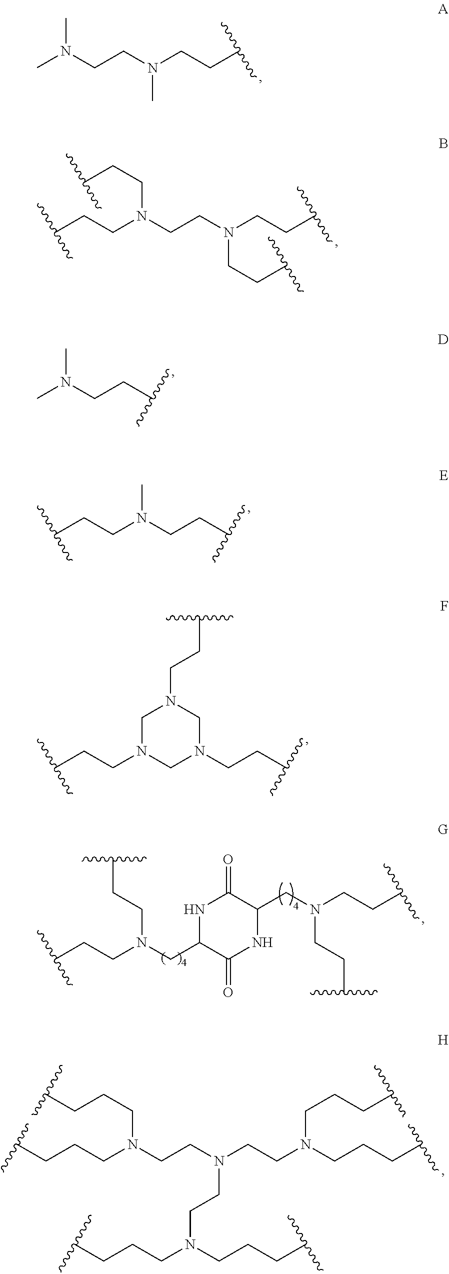

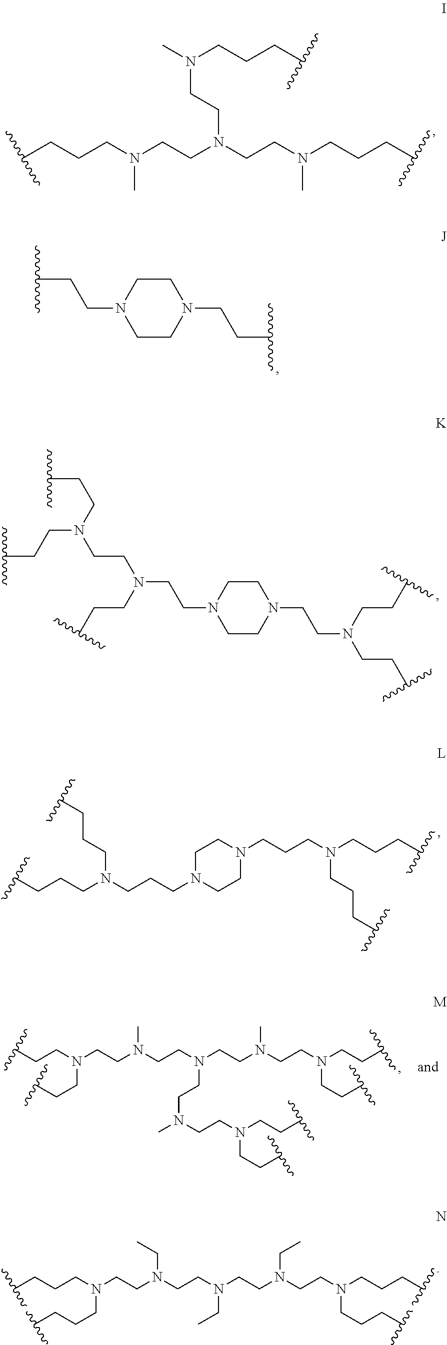 [SMILES: *CCCN(C)CCN(CCN(C)CCC*)CCN(C)CCC* ; *CCN1CCN(CC*)CC1 ; *CCN(CC*)CCN(CC*)CCN1CCN(CCN(CC*)CC*)CC1 ; *CCCN(CCC*)CCCN1CCN(CCCN(CCC*)CCC*)CC1 ; *CCN(CC*)CCN(C)CCN(CCN(C)CCN(CC*)CC*)CCN(C)CCN(CC*)CC* ; *CCCN(CCC*)CCN(CC)CCN(CC)CCN(CC)CCN(CCC*)CCC*]

7-19. (canceled)

20. The compound of claim 1, wherein R₁ has a structure selected from (CL), (DD) and (TD):

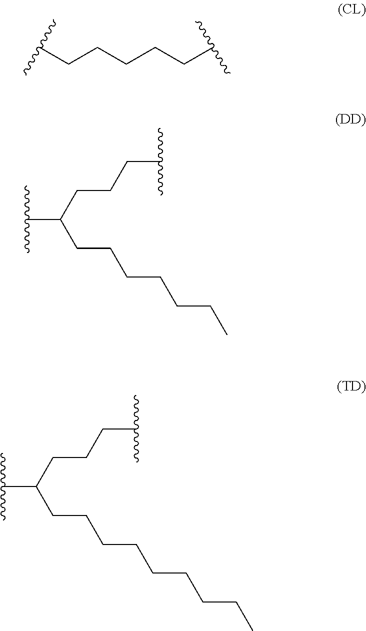 [SMILES: *CCCCC* ; *CCCC(*)CCCCCCC ; *CCCC(*)CCCCCCCCC]

21-28. (canceled)

29. The compound of claim 1, wherein R₂ is hydrogen or C₁₋₆ alkyl; and R₃ is selected from:

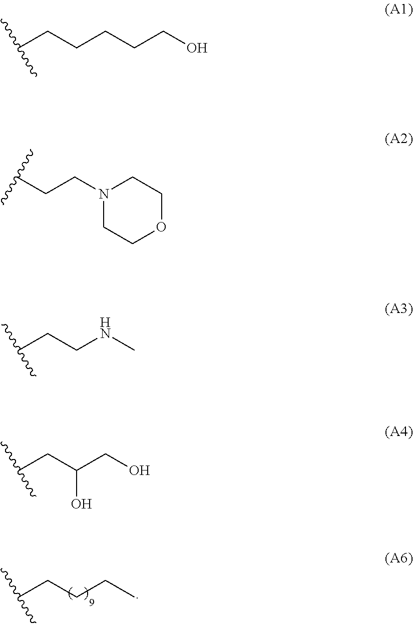

30. The compound of claim 1, wherein n is an integer between 1 and 10, inclusive.

31. (canceled)

32. The compound of claim 1, wherein m an integer between 1 and 20, inclusive.

33. (canceled)

34. The compound of claim 1, wherein R, X, R₁, n and m are defined as follows:

| (II) | R | X | R₁ | n | m |
|---|---|---|---|---|---|
| IDD3 | 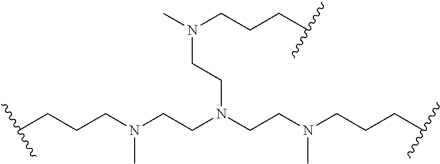 [SMILES: *CCCN(C)CCN(CCN(C)CCC*)CCN(C)CCC*] | O | 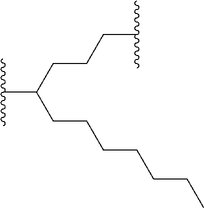 [SMILES: *CCCC(*)CCCCCCC] | 3 | 3 |
| ITD3 | 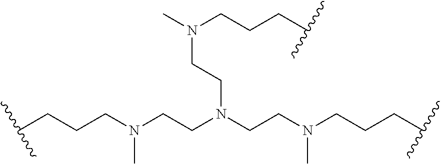 [SMILES: *CCCN(C)CCN(CCN(C)CCC*)CCN(C)CCC*] | O | 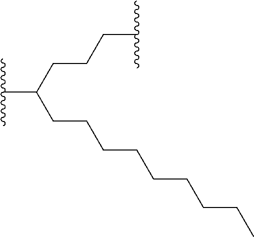 [SMILES: *CCCC(*)CCCCCCCCC] | 3 | 3 |
| ATD3 | 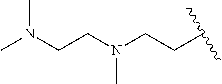 [SMILES: *CCN(C)CCN(C)C] | O | 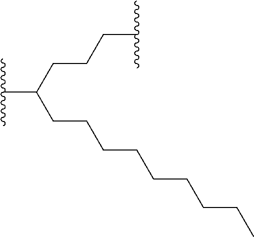 [SMILES: *CCCC(*)CCCCCCCCC] | 3 | 1 |
| BDD3 | 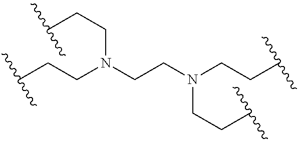 [SMILES: *CCN(CC*)CCN(CC*)CC*] | O | 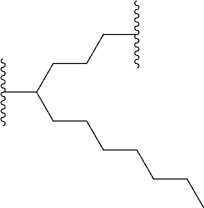 [SMILES: *CCCC(*)CCCCCCC] | 3 | 4 |
| ADD5 | 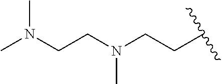 [SMILES: *CCN(C)CCN(C)C] | O | 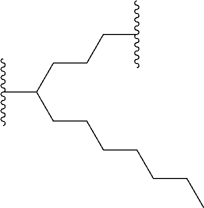 [SMILES: *CCCC(*)CCCCCCC] | 5 | 1 |
| BTD3 | 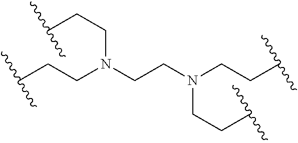 [SMILES: *CCN(CC*)CCN(CC*)CC*] | O | 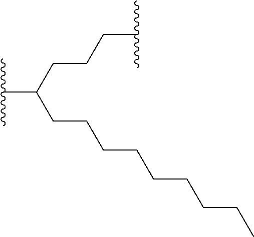 [SMILES: *CCCC(*)CCCCCCCCC] | 3 | 4 |
| HTD3 | 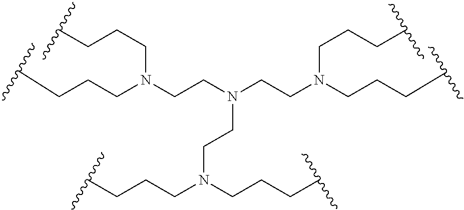 [SMILES: *CCCN(CCC*)CCN(CCN(CCC*)CCC*)CCN(CCC*)CCC*] | O | 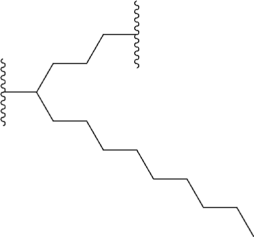 [SMILES: *CCCC(*)CCCCCCCCC] | 3 | 6 |
| BTD5 | 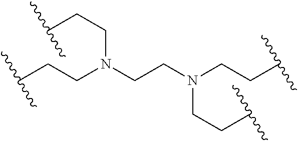 [SMILES: *CCN(CC*)CCN(CC*)CC*] | O | 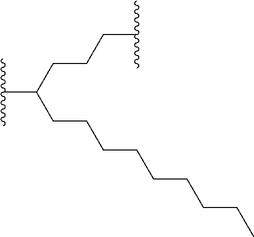 [SMILES: *CCCC(*)CCCCCCCCC] | 5 | 4 |
| BDD5 | 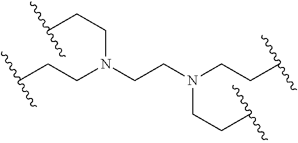 [SMILES: *CCN(CC*)CCN(CC*)CC*] | O | 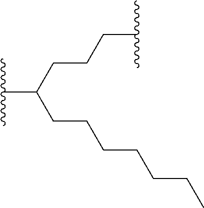 [SMILES: *CCCC(*)CCCCCCC] | 5 | 4 |
| DTD5 | 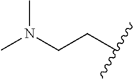 [SMILES: *CCN(C)C] | O | 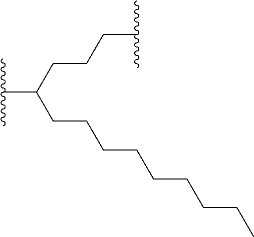 [SMILES: *CCCC(*)CCCCCCCCC] | 5 | 1 |
| ETD5 | 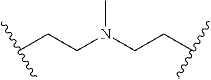 [SMILES: *CCN(C)CC*] | O | 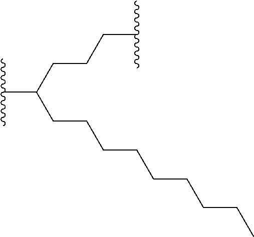 [SMILES: *CCCC(*)CCCCCCCCC] | 5 | 2 |
| FDD5 | 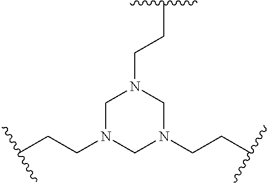 [SMILES: *CCN1CN(CC*)CN(CC*)C1] | O | 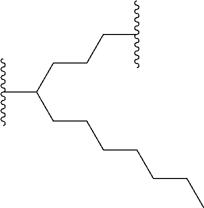 [SMILES: *CCCC(*)CCCCCCC] | 5 | 3 |

35. The compound of claim 1, wherein R, X, R₁, R₂, R₃, n and m are defined as follows:

| (III) | R | X | R₁ | R₂ | R₃ | n | m |
|---|---|---|---|---|---|---|---|
| BDD3-Ac1 | 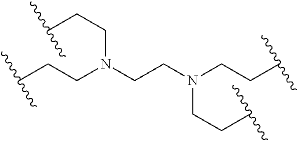 [SMILES: *CCN(CC*)CCN(CC*)CC*] | O | 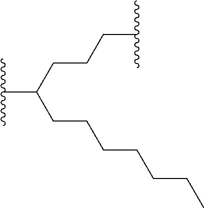 [SMILES: *CCCC(*)CCCCCCC] | H | 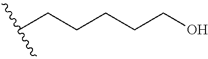 [SMILES: *CCCCCO] | 3 | 4 |
| BDD3-Ac2 | 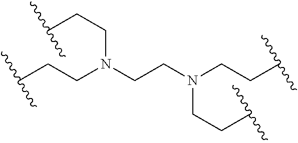 [SMILES: *CCN(CC*)CCN(CC*)CC*] | O | 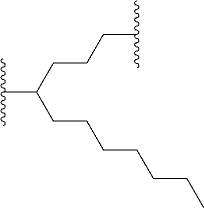 [SMILES: *CCCC(*)CCCCCCC] | H | 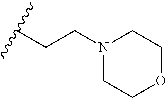 [SMILES: *CCN1CCOCC1] | 3 | 4 |
| BDD3-Ac3 | 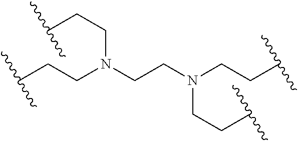 [SMILES: *CCN(CC*)CCN(CC*)CC*] | O | 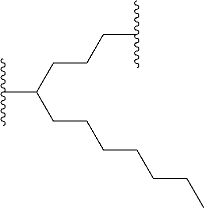 [SMILES: *CCCC(*)CCCCCCC] | H | 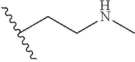 [SMILES: *CCNC] | 3 | 4 |
| BDD3-Ac4 | 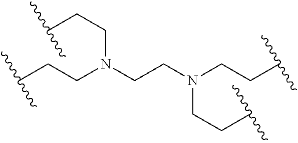 [SMILES: *CCN(CC*)CCN(CC*)CC*] | O | 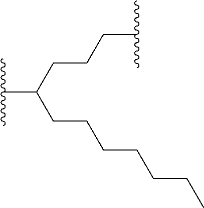 [SMILES: *CCCC(*)CCCCCCC] | H | 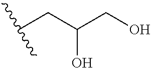 [SMILES: *CC(O)CO] | 3 | 4 |
| BDD3-Ac6 | 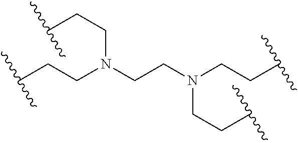 [SMILES: *CCN(CC*)CCN(CC*)CC*] | O | 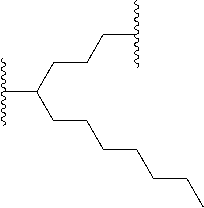 [SMILES: *CCCC(*)CCCCCCC] | H | 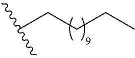 | 3 | 4 |

36. The compound of claim 35, having the structure:

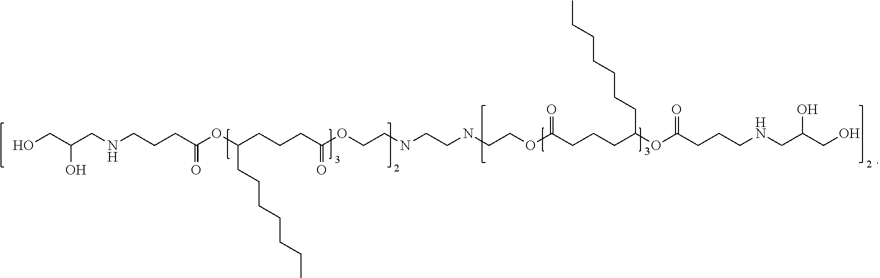

37. The compound of claim 35, having the structure:

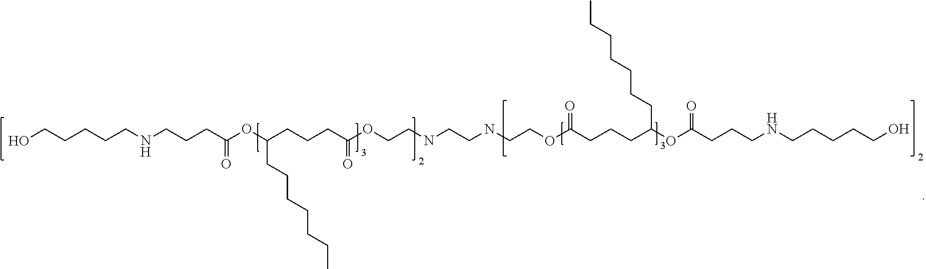

38. A method of making a compound of Formula (II), as defined in claim 2, or a salt thereof: 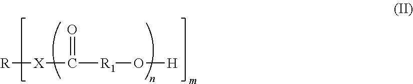
comprising acylating a compound of Formula (Ib), or a salt thereof:
R(X)ₘ (Ib)
with a compound of Formula (Ic): 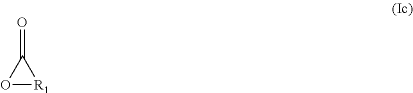
to obtain a compound of Formula (II).

39. A method of making a compound of Formula (III), as defined in claim 3, or a salt thereof: 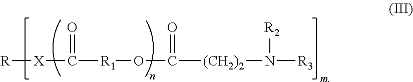
comprising:
acylating a compound of Formula (II): 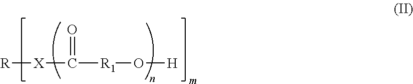
to obtain a compound of Formula (IIa): 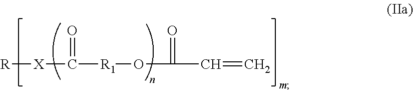
and
alkylating a compound of Formula (lib): 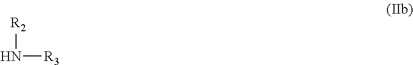
with a compound of Formula (IIa) to obtain the compound of Formula (III), or a salt thereof.

40-41. (canceled)

42. A composition comprising a compound of claim 1 and an excipient.

43-47. (canceled)

48. The composition of claim 42, wherein the composition further comprises an agent.

49. (canceled)

50. The composition of claim 48, wherein the agent is a polynucleotide.

51-60. (canceled)

61. A method of treating a disease, disorder, or condition from which a subject suffers, comprising administering to a subject in need thereof an effective amount of a composition of claim 48.

62. (canceled)

63. A method of delivering a polynucleotide to a cell, comprising contacting the cell with a composition of claim 50.

64-66. (canceled)
